(19) United States
(12) Patent Application Publication
(10) Pub. No.: US 2004/0170630 A1
(43) Pub. Date: Sep. 2, 2004

(54) Human monoclonal antibodies to heparanase

(76) Inventors: Haichun Huang, Fremont, CA (US); Steven Holmes, Great Chishill (GB); Sean Mason, Cambridge (GB)

Correspondence Address: LAHIVE & COCKFIELD, LLP., 28 STATE STREET, BOSTON, MA, 02109, US

(21) Appl. No.: 10/703,714

(22) Filed: Nov. 7, 2003

Related U.S. Application Data
(60) Provisional application No. 60/424,803, filed on Nov. 7, 2002.

(51) Int. Cl. A61K 39/395; C07K 16/40

(52) U.S. Cl. 424/146.1; 530/388.26

(57) ABSTRACT

Isolated human monoclonal antibodies which bind to and inhibit human heparanase, and related antibody-based compositions and molecules, are disclosed. The human antibodies can be produced by a transfectoma or in a nonhuman transgenic animal, e.g., a transgenic mouse, capable of producing multiple isotypes of human monoclonal antibodies by undergoing V-D-J recombination and isotype switching. Also disclosed are pharmaceutical compositions comprising the human antibodies, nonhuman transgenic animals and hybridomas which produce the human antibodies, and therapeutic and diagnostic methods for using the human antibodies.

### BACKGROUND OF THE INVENTION

Heparanase is an enzyme that can degrade heparan sulphate, heparin proteoglycans (HPG), and heparan sulphate proteoglycans (HSPG). Heparanase activity in mammalian cells is well known. The activity has been identified in various melanoma cells (Nakajima, et al. (1986) Cancer Letters 31:277-283), mammary adenocarcinoma cells (Parish, et al. (1987) Int. J. Cancer 40:511-518), leukemic cells (Yahalom, et al. (1988) Leukemia Research 12:711-717), prostate carcinoma cells (Kosir, et al. (1997) J. Surg. Res. 67:98-105), mast cells (Ogren and Lindahl (1975) J. Biol. Chem. 250:2690-2697), macrophages (Savion, et al. (1987) J. Cell. Physiol. 130:85-92), mononuclear cells (Sewell, et al. (1989) Biochem. J. 264:777-783), neutrophils (Matzner, et al.(1992) 51:519-524), T-cells (Vettel et al. (1991) Eur. J. Immunol. 21:2247-2251), platelets (Haimovitz-Friedman, et al.(1991) Blood 78:789-796), endothelial cells (Godder, et al. (1991) J. Cell Physiol. 148:274-280), and placenta (Klein and von Figura (1976) BBRC 73:569).

Elevated heparanase activity has been documented in mobile, invasive cells, such as metastatic tumor cells. Examples include invasive melanoma (Nakajima, et al. (1983) Science 220:611), lymphoma (Vlodavsky et al. (1983) Cancer Res. 43: 2704), fibrosarcoma (Becker et al. (1986) J. Natl. Cancer Inst., 77:417), rhabdomyosarcoma (U.S. Pat. No. 4,882,318), mastocytoma, mammary adeno-carcinoma, leukemia, and rheumatoid fibroblasts. Heparanase activity has also been documented in non-pathologic situations involving the migration of lymphocytes, neutrophils, macrophages, eosinophils and platelets (Vlodavsky et al. (1992) Invasion Metastasis 12:112-127). Heparanase activity is also implicated in inflammation (Hoogewerf J. (1995) Biol. Chem. 270:3268-3277; WO97/11684), wound healing (Whitelock et al. (1996) J. Biol. Chem. 271: 10079-10086), angiogenesis (U.S. Pat. No. 5,567,417), inflammatory diseases such as arthritis (including rheumatoid arthritis and osteoarthritis), asthma, lupus erythematosus, allografts, as well as vascular restenosis, atherosclerosis, tumor growth and progression, fibro-proliferative disorders, Alzheimer's Disease (McBubbin, et al. (1999) Biochem. J. 256:775-783); Snow et al. (1996) Neuron 12: 219-234). In general, heparanase activity is present in mobile invasive cells in a variety of pathologies. In fact, raised levels of carbohydrate processing enzymes, such as heparanases, secreted by tumor cells correlates to metastatic potential (e.g., Vlodavsky, et al. (1994) Invasion Metastasis 14:290-302; and (1999) Nature Medicine 5:793-802).

Carbohydrates represent a large fraction of the total mass of all extracellular matrices (ECM). Tumor cells secrete large quantities of carbohydrate degrading enzymes as they penetrate the ECM. The carbohydrate fragments generated by glycosidase action also promote the cancer phenotype since many are growth-stimulatory. For example, heparanase activity can release heparan sulfate fragments which can increase the potency of a variety of growth factors and can elicit cell growth stimulation when they are bound by the appropriate cell surface receptors (Folkman and Shing (1992) Adv. Exp. Med. Biol. 313:355-64).

While inhibitors of heparanase may be valuable in the treatment of a wide variety of disorders, it is known that murine antibodies do not constitute ideal therapeutic agents. Moreover, treatment with murine antibodies generally triggers severe immune reactions in patients. Accordingly, the need exists for improved therapeutic antibodies against heparanase which are effective at treating and/or preventing diseases related to overexpression of heparanase.

### SUMMARY OF THE INVENTION

The present invention provides improved antibody therapeutics for treating and preventing diseases mediated by heparanase, including tumor-related diseases. The antibodies are improved in that they are fully human and, thus, are less immunogenic in patients. Furthermore, the antibodies display high affinity for heparanase and have other beneficial functional properties. The antibodies are also therapeutically useful in that they inhibit heparanase activity which is involved in a variety of diseases.

In one embodiment, the present invention provides isolated human monoclonal antibodies which specifically bind to and inhibit human heparanase, as well as compositions containing one or a combination of such antibodies. The human antibodies reduce the activity of human heparanase, for example, in mediating tumor cell migration and/or growth. Accordingly, human monoclonal antibodies of the invention can be used as diagnostic and/or therapeutic agents in vivo and in vitro.

Human antibodies of the invention include IgG1 (e.g., IgG1k) antibodies having an IgG1 heavy chain and a kappa light chain. However, other antibody isotypes are also encompassed by the invention, including IgG2, IgG3, IgG4, IgM, IgA1, IgA2, IgAsec, IgD, and IgE. The antibodies can be whole antibodies or antigen-binding fragments thereof including, for example, Fab, F(ab′)₂, Fv and single chain Fv fragments.

In a particular embodiment, the invention provides a human antibody referred to as 22D9 encoded by human heavy chain and human kappa light chain nucleic acids comprising nucleotide sequences in their variable regions as set forth in SEQ ID NO:5 and SEQ ID NO:7, respectively, and conservative sequence modifications thereof. In another embodiment, human antibody 22D9 is characterized as having a human heavy chain and human kappa light chain variable regions comprising the amino acid sequences as set forth in SEQ ID NO:6 and SEQ ID NO:8, respectively, and conservative sequence modifications thereof.

Other particular human antibodies of the invention include those referred to as 2H8, 13B2, 5G10 and 1E7, encoded by human IgG heavy chain and human kappa light chain nucleic acids comprising nucleotide sequences in their variable regions selected from the group consisting of SEQ ID NOs:1, 9, 13, and 17 and SEQ ID NOs:3, 11, 15, and 19, respectively, and conservative sequence modifications thereof. These antibodies also include IgG heavy chain and kappa light chain variable regions which comprise amino acid sequences selected from the group consisting of SEQ ID NOs:2, 10, 14, and 18 and SEQ ID NOs:4, 12, 16, and 20, respectively, and conservative sequence modifications thereof.

Still other particular human antibodies of the invention include those which comprise a CDR domain having a human heavy and light chain CDR1 region, a human heavy and light chain CDR2 region, and a human heavy and light chain CDR3 region, wherein

(a) the CDR1, CDR2, and CDR3 of the human heavy chain regions comprise an amino acid sequence selected from the group consisting of the amino acid sequences of the CDR1, CDR2, and CDR3 regions shown in FIG. 1 (SEQ ID NOs:25, 26, or 27), FIG. 3 (SEQ ID NOs:31, 32, or 33), FIG. 5 (SEQ ID NOs:37, 38, or 39), FIG. 7 (SEQ ID NOs:43, 44, or 45), and FIG. 9 (SEQ ID NOs:49, 50, or 51), and conservative sequence modifications thereof, and

(b) the CDR1, CDR2, and CDR3 of the human light chain regions comprise an amino acid sequence selected from the group consisting of the amino acid sequences of the CDR1, CDR2, and CDR3 regions shown in FIG. 2 (SEQ ID NOs:28, 29, or 30), FIG. 4 (SEQ ID NOs:34, 35, or 36), FIG. 6 (SEQ ID NOs:40, 41, or 42), FIG. 8 (SEQ ID NOs:46, 47, or 48), and FIG. 10 (SEQ ID NOs:52, 53, or 54), and conservative sequence modifications thereof.

Human antibodies of the invention can be produced recombinantly in a host cell, such as a transfectoma (e.g., a transfectoma consisting of immortalized CHO cells or lymphocytic cells) containing nucleic acids encoding the heavy and light chains of the antibody, or be obtained directly from a hybridoma which expresses the antibody (e.g., which includes a B cell obtained from a transgenic nonhuman animal, e.g., a transgenic mouse, having a genome comprising a human heavy chain transgene and a human light chain transgene that encode the antibody, fused to an immortalized cell). In a particular embodiment, the antibodies are produced by a hybridoma selected from the group consisting of hybridomas 2H8, 22D9, 13B2, 5G10, and 1E7, or by a host cell (e.g., a CHO cell) transfectoma containing human heavy chain and human light chain nucleic acids which comprise nucleotide sequences in their variable regions selected from the group consisting of SEQ ID NOs: 1, 5, 9, 13, and 17, and SEQ ID NOs: 3, 7, 11, 15, and 19, respectively, and conservative modifications thereof.

In another embodiment, human anti-heparanase antibodies of the present invention can be characterized by one or more of the following properties:

a) specificity for human heparanase;

b) a binding affinity to heparanase (K_{A}) of at least about 10⁷ M⁻¹, preferably about, 10⁸ M⁻¹, and more preferably, about 10⁹ M⁻¹ to 10¹⁰ M⁻¹ or higher;

c) an association rate constant (K_{assoc}) to heparanase of at least about 10⁻⁴ M⁻¹S⁻¹, preferably about 10⁻⁵ M⁻¹S⁻¹ or higher;

d) a dissociation rate constant (K_{diss}) from heparanase of about 10⁻³ S^{-1 1}or less, preferably about 10⁻⁴ S⁻¹ or less, more preferably, 10⁻⁵ S⁻¹ or less;

e) the ability to inhibit the enzymatic activity of heparanase in a dose-dependent manner at pH 5.2 and/or pH 7.4; and/or

f) the ability to inhibit tumor cell growth and/or motility induced by human heparanase.

Examples of tumor cells which can be inhibited by human antibodies of the present invention include, but are not limited to, melanoma cells, lymphoma cells, fibrosarcoma cells, rhabdomyosarcoma cells, mastocytoma cells, mammary adenocarcinoma cells, leukemia cells, prostate carcinoma cells, pancreatic carcinoma cells, bladder carcinoma cells, mast cells, and rheumatoid fibroblasts. Other cells whose function or activity can be inhibited by human antibodies of the present invention include mononuclear cells, T-cells, endothelial cells, lymphocytes, neutrophils, macrophages, and eosinophils involved in diseases such as asthma, lupus erythematosus, and allograft rejection.

In another aspect, the present invention provides nucleic acid molecules encoding human antibodies, or antigen-binding portions thereof, of the invention. Recombinant expression vectors which include nucleic acids encoding antibodies of the invention, and host cells transfected with such vectors, are also encompassed by the invention, as are methods of making the antibodies of the invention by culturing such host cells, e.g., an expression vector comprising a nucleotide sequence encoding the variable and constant regions of the heavy and light chains of antibodies 2H8, 22D9, 13B2, 5G10, or 1E7 produced by the hybridoma.

In yet another aspect, the invention provides isolated B-cells from a transgenic nonhuman animal, e.g., a transgenic mouse, which express human anti-heparanase antibodies of the invention. Preferably, the isolated B cells are obtained from a transgenic nonhuman animal, e.g., a transgenic mouse, which has been immunized with a purified or recombinant preparation of heparanase antigen and/or cells expressing heparanase. In one embodiment, the transgenic nonhuman animal, e.g., a transgenic mouse, has a genome comprising a human heavy chain transgene or transchromosome and a human light chain transgene encoding all or a portion of an antibody of the invention. The isolated B-cells can then be immortalized to provide a source (e.g., a hybridoma) of human anti-heparanase antibodies.

Accordingly, the present invention also provides a hybridoma capable of producing human monoclonal antibodies of the invention that specifically bind to heparanase. In one embodiment, the hybridoma includes a B cell obtained from a transgenic nonhuman animal, e.g., a transgenic mouse, having a genome comprising a human heavy chain transgene and a human light chain transgene encoding all or a portion of an antibody of the invention, fused to an immortalized cell. Particular hybridomas of the invention include 2H8, 22D9, 13B2, 5G10, and 1E7.

In yet another aspect, the invention provides a transgenic nonhuman animal, such as a transgenic mouse, which expresses human monoclonal antibodies that specifically bind to heparanase. In a particular embodiment, the transgenic nonhuman animal is a transgenic mouse having a genome comprising a human heavy chain transgene or transchromosome and a human light chain transgene encoding all or a portion of an antibody of the invention. Suitable transgenic mice include, for example HuMAb mice and KM mice described herein. The transgenic nonhuman animal can be immunized with a purified or recombinant preparation of heparanase antigen and/or cells expressing heparanase. Preferably, the transgenic nonhuman animal, e.g., the transgenic mouse, is capable of producing multiple isotypes of human monoclonal antibodies to heparanase (e.g., IgG, IgA and/or IgM) by undergoing V-D-J recombination and isotype switching. Isotype switching may occur by, e.g., classical or non-classical isotype switching.

In another aspect, the present invention provides methods for producing human monoclonal antibodies which specifically bind to heparanase. In one embodiment, the method includes immunizing a transgenic nonhuman animal, e.g., a transgenic mouse, having a genome comprising a human heavy chain transgene or transchromosome and a human light chain transgene encoding all or a portion of an antibody of the invention, with a purified or enriched preparation of heparanase antigen and/or cells expressing heparanase. B cells (e.g., splenic B cells) of the animal can then be obtained and fused with myeloma cells to form immortal hybridoma cells that secrete human monoclonal antibodies against heparanase.

In yet another aspect, human anti-heparanase antibodies of the invention are derivatized, linked to or co-expressed with another functional molecule, e.g., another peptide or protein. For example, a human antibody or antigen-binding portion of the invention can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, the present invention encompasses a large variety of antibody conjugates, bi- and multispecific molecules, and fusion proteins, all of which bind to heparanase and to other molecules or cells.

In another aspect, the present invention provides a conjugate comprising a human anti-heparanase antibody of the invention linked to a therapeutic moiety, e.g., a cytotoxic drug, an enzymatically active toxin, or a fragment thereof, a radioisotope, a small molecule anti-cancer drug, an anti-inflammatory agent, or an immunosuppressive agent. Alternatively, human antibodies of the invention can be co-administered separately with such therapeutic and cytotoxic agents (i.e., not linked to them).

In another aspect, the present invention provides compositions, e.g., pharmaceutical and diagnostic compositions/kits, comprising a pharmaceutically acceptable carrier and at least one human monoclonal antibody of the invention, or a combination of antibodies, preferably each of which binds to a distinct epitope.

In yet another aspect, the invention provides a method for inhibiting tumor cell proliferation and/or motility induced by human heparanase by administering one or more human antibodies of the invention. In one embodiment, the method comprises contacting a tumor cell either in vitro or in vivo with one or a combination of human monoclonal antibodies of the invention. The method can be employed in culture, e.g. in vitro or ex vivo (e.g., cultures comprising tumor cells). Alternatively, the method can be performed in a subject, e.g., as part of an in vivo (e.g., therapeutic or prophylactic) protocol.

For use in in vivo treatment and prevention of diseases related to heparanase expression (e.g., over-expression), human antibodies of the invention can be administered to patients (e.g., human subjects) at therapeutically effective dosages (e.g., dosages which result in tumor growth inhibition and/or tumor cell migration inhibition) using any suitable route of administration, such as injection and other routes of administration known in the art for antibody-based clinical products.

Heparanase-related diseases which can be treated and/or prevented using the human antibodies of the invention include, but are not limited to, cancers, inflammatory disorders, and autoimmune diseases. For example, cancers which can be treated and/or prevented include melanomas, lymphomas, prostate cancer, pancreatic cancer, bladder cancer, fibrosarcomas, rhabdomyosarcomas, mastocytomas, mammary adenocarcinomas, leukemias, and rheumatoid fibroblastomas. Autoimmune diseases which can be treated include, for example, arthritis, e.g., rheumatoid arthritis or and osteoarthritis, asthma, lupus erythematosus, allograft rejection, vascular restenosis, and atherosclerosis. Another disease which can be treated and/or prevented is Alzheimer's disease.

In yet another aspect, the present invention provides a method for detecting in vitro or in vivo the presence of heparanase antigen in a sample, e.g., for diagnosing an heparanase-related disease. In one embodiment, this is achieved by contacting a sample to be tested, optionally along with a control sample, with a human monoclonal antibody of the invention (or an antigen-binding portion thereof) under conditions that allow for formation of a complex between the antibody and heparanase. Complex formation is then detected (e.g., using an ELISA). When using a control sample along with the test sample, complex is detected in both samples and any statistically significant difference in the formation of complexes between the samples is indicative of the differential presence of heparanase antigen in the test sample.

Other features and advantages of the instant invention will be apparent from the following detailed description and claims.

### BRIEF DESCRIPTION OF THE DRAWINGS

FIG. 1 shows the nucleotide sequence (SEQ ID NO:1) and corresponding amino acid sequence (SEQ ID NO:2) of the heavy chain V region of human monoclonal antibody 2H8 with CDR regions designated.

FIG. 2 shows the nucleotide sequence (SEQ ID NO:3) and corresponding amino acid sequence (SEQ ID NO:4) of the light (kappa) chain V region of human monoclonal antibody 2H8 with CDR regions designated.

FIG. 3 shows the nucleotide sequence (SEQ ID NO:5) and corresponding amino acid sequence (SEQ ID NO:6) of the heavy chain V region of human monoclonal antibody 22D9 with CDR regions designated.

FIG. 4 shows the nucleotide sequence (SEQ ID NO:7) and corresponding amino acid sequence (SEQ ID NO:8) of the light (kappa) chain V region of human monoclonal antibody 22D9 with CDR regions designated.

FIG. 5 shows the nucleotide sequence (SEQ ID NO:9) and corresponding amino acid sequence (SEQ ID NO:10) of the heavy chain V region of human monoclonal antibody 13B2 with CDR regions designated.

FIG. 6 shows the nucleotide sequence (SEQ ID NO:11) and corresponding amino acid sequence (SEQ ID NO:12) of the light (kappa) chain V region of human monoclonal antibody 13B2 with CDR regions designated.

FIG. 7 shows the nucleotide sequence (SEQ ID NO:13) and corresponding amino acid sequence (SEQ ID NO:14) of the heavy chain V region of human monoclonal antibody 5G10 with CDR regions designated.

FIG. 8 shows the nucleotide sequence (SEQ ID NO:15) and corresponding amino acid sequence (SEQ ID NO:16) of the light (kappa) chain V region of human monoclonal antibody 5G10 with CDR regions designated.

FIG. 9 shows the nucleotide sequence (SEQ ID NO:17) and corresponding amino acid sequence (SEQ ID NO: 18) of the heavy chain V region of human monoclonal antibody 1E7 with CDR regions designated.

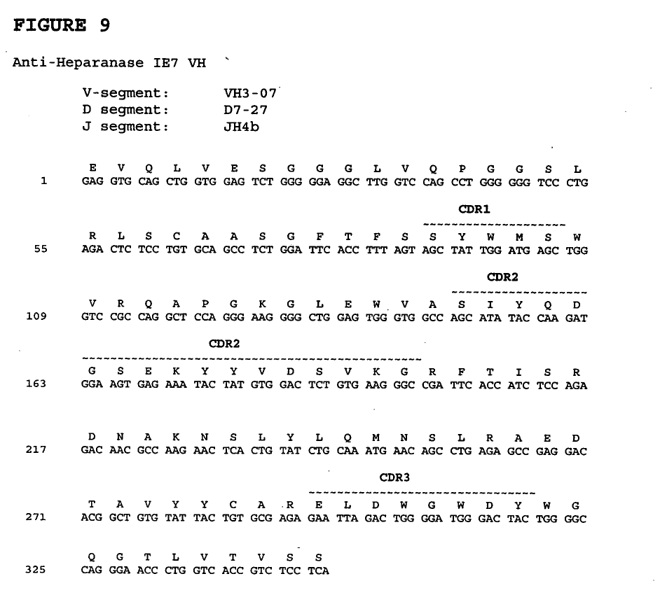

FIG. 10 shows the nucleotide sequence (SEQ ID NO:19) and corresponding amino acid sequence (SEQ ID NO:20) of the light (kappa) chain V region of human monoclonal antibody 1E7 with CDR regions designated.

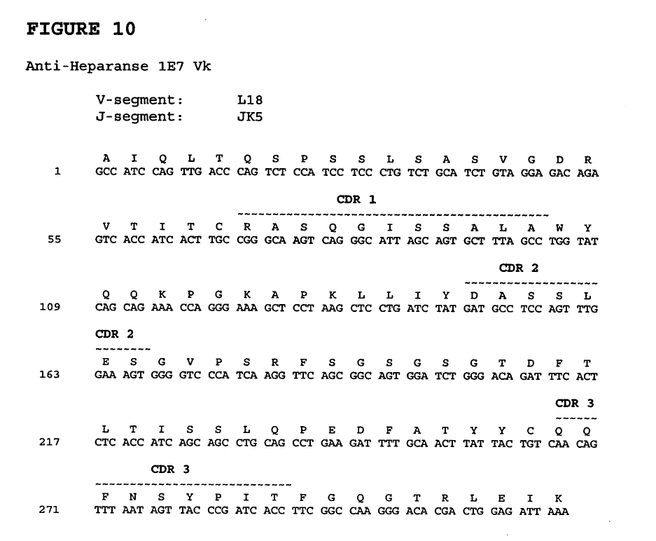

FIG. 11 shows the amino acid sequence of the heavy chain V region of human monoclonal antibodies 22D9 (SEQ ID NO:6), 2H8 (SEQ ID NO:2), and 5G10 (SEQ ID NO:14) compared to the germline sequence (SEQ ID NO:21).

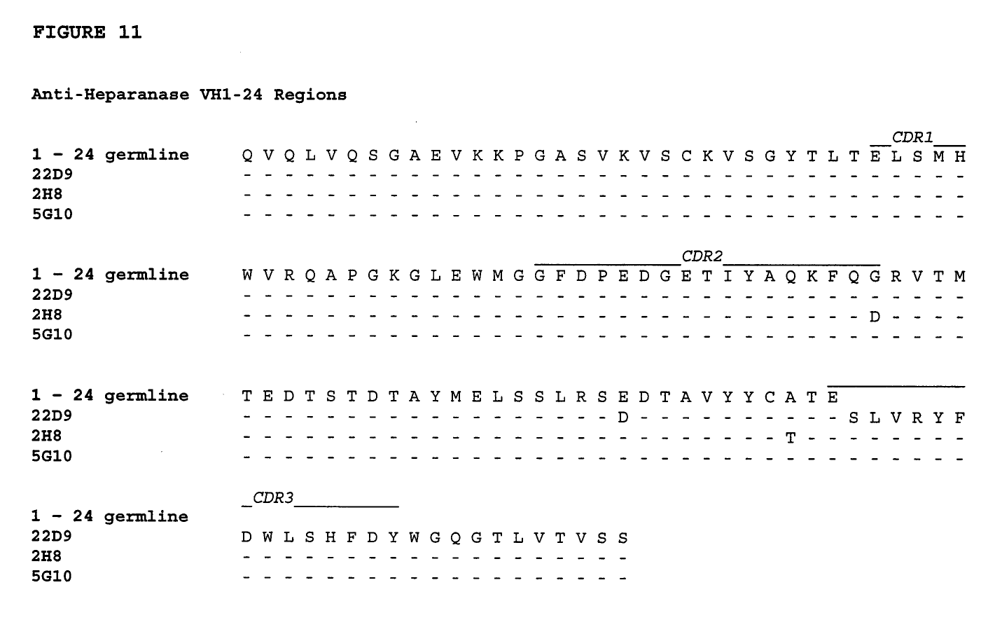

FIG. 12 shows the amino acid sequence of the heavy chain V region of human monoclonal antibodies 13B2 (SEQ ID NO:10) and 1E7 (SEQ ID NO:18) compared to the germline sequence (SEQ ID NO:22).

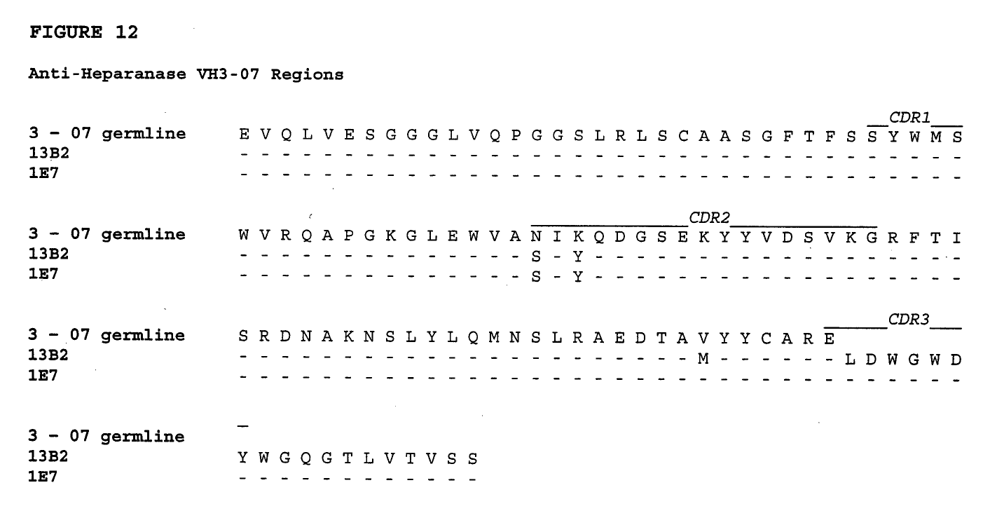

FIG. 13 shows the amino acid sequence of the light (kappa) chain V region of human monoclonal antibodies 22D9 (SEQ ID NO:8), 2H8 (SEQ ID NO:4), and 5G10 (SEQ ID NO:16) compared to the germline sequence (SEQ ID NO:23).

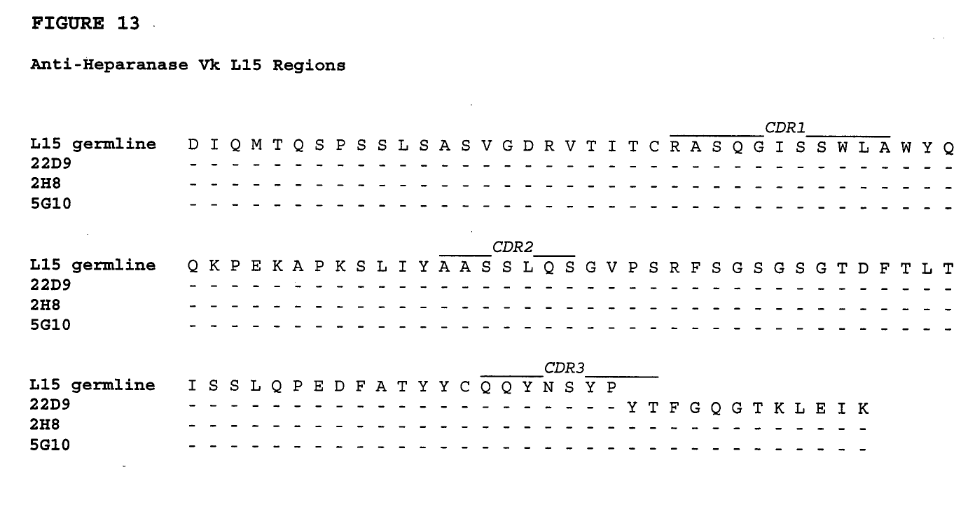

FIG. 14 shows the amino acid sequence of the light (kappa) chain V region of human monoclonal antibodies 13B2 (SEQ ID NO:12) and 1E7 (SEQ ID NO:20) compared to the germline sequence (SEQ ID NO:24).

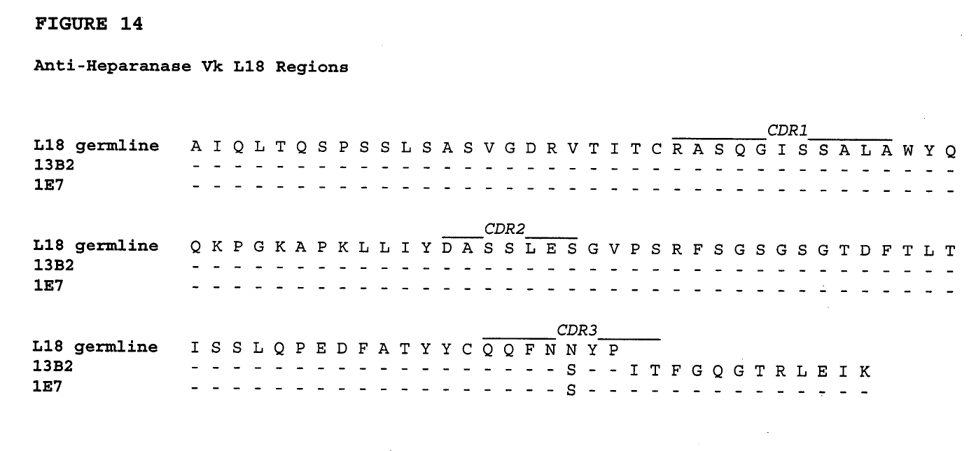

FIG. 15 is a graph showing the activity of heparanase at pH5.2 and 7.4.

FIG. 16 is a graph showing that human monoclonal antibody 22D9 inhibits heparanase activity in a dose dependent manner at pH5.2.

FIG. 17 is a graph showing comparable inhibition of heparanase activity with human monoclonal antibody 22D9 at pH5.2 and 7.4.

FIG. 18 is a graph showing the effect of human monoclonal antibody 22D9 on the growth of MDA-MB 435 cells in nu/nu mice.

### DETAILED DESCRIPTION OF THE INVENTION

The present invention provides novel antibody-based therapeutics for treating and diagnosing a variety of disorders mediated by heparanase (i.e., disorders caused by the activity of heparanase). As used herein, the term “activity of heparanase” includes any cellular response induced by heparanase, e.g., induced by heparanase-mediated enzymatic degradation of glycosaminoglycans into fragments. Such cellular responses include, for example, tumor cell invasion, metastasis, and angiogenesis. Other cellular responses associated with heparanase activity include inflammatory or autoimmune responses.

Therapies of the invention employ isolated human monoclonal antibodies which specifically bind to an epitope present on heparanase. Other isolated human monoclonal antibodies encompassed by the present invention include IgA, IgG1-4, IgE, IgM, and IgD antibodies. In one embodiment, the human antibodies are produced in a nonhuman transgenic animal, e.g., a transgenic mouse, capable of producing multiple isotypes of human monoclonal antibodies to heparanase (e.g., IgG, IgA and/or IgE) by undergoing V-D-J recombination and isotype switching. Accordingly, aspects of the invention include not only antibodies, antibody fragments, and pharmaceutical compositions thereof, but also nonhuman transgenic animals, B-cells, host cell transfectomas, and hybridomas which produce monoclonal antibodies. Methods of using the antibodies of the invention to detect a cell expressing heparanase or to inhibit tumor cell growth and/or motility induced by human heparanase, either in vitro or in vivo, are also encompassed by the invention.

In order that the present invention may be more readily understood, certain terms are first defined. Additional definitions are set forth throughout the detailed description.

The terms “heparanase” and “heparanase antigen” are used interchangeably herein, and include all known variants, isoforms and species homologs of human heparanase. Examples of human heparanase include the protein of Genbank Accession No. NP-006656 and the protein encoded by Genbank Accession No. NM-006665.

As used herein, the term “inhibits growth” (e.g., referring to cells) is intended to include any measurable decrease in the growth of a cell when contacted with an anti-heparanase antibody as compared to the growth of the same cell not in contact with an anti-heparanase antibody, e.g., the inhibition of growth of a cell by at least about 10%, 20%, 30%, 40%, 50%, 60%, 70%, 80%, 90%, 99%, or 100%.

The term “antibody” as referred to herein includes whole antibodies and any antigen binding fragment (i.e., “antigen-binding portion”) or single chain thereof. A “whole antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., heparanase). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are whole antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “bispecific molecule” is intended to include any agent, e.g., a protein, peptide, or protein or peptide complex, which has two different binding specificities. For example, the molecule may bind to, or interact with, (a) a cell surface antigen and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with, (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to cell surface antigens, such as heparanase, and to other targets, such as Fc receptors on effector cells.

The term “bispecific antibodies” also includes diabodies. Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

The term “human antibody derivatives” refers to any modified form of the antibody, e.g., a conjugate of the antibody and another agent or antibody.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library, and wherein the selected human antibody is at least 90%, more preferably at least 95%, even more preferably at least 96%, 97%, 98%, or 99% identical in amino acid sequence to the amino acid sequence encoded by the germline immunoglobulin gene. Typically, a human antibody derived from a particular human germline sequence will display no more than 10 amino acid differences, more preferably, no more than 5, or even more preferably, no more than 4, 3, 2, or 1 amino acid difference from the amino acid sequence encoded by the germline immunoglobulin gene.

As used herein, the term “heteroantibodies” refers to two or more antibodies, antibody binding fragments (e.g., Fab), derivatives therefrom, or antigen binding regions linked together, at least two of which have different specificities. These different specificities include a binding specificity for an Fc receptor on an effector cell, and a binding specificity for an antigen or epitope on a target cell, e.g., a tumor cell. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic or transchromosomal nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further in Section I, below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, a “heterologous antibody” is defined in relation to the transgenic nonhuman organism producing such an antibody. This term refers to an antibody having an amino acid sequence or an encoding nucleic acid sequence corresponding to that found in an organism not consisting of the transgenic nonhuman animal, and generally from a species other than that of the transgenic nonhuman animal.

As used herein, a “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies, discussed supra.

An “isolated antibody,” as used herein, is intended to refer to an antibody which is substantially free of other antibodies having different antigenic specificities (e.g., an isolated antibody that specifically binds to heparanase is substantially free of antibodies that specifically bind antigens other than heparanase). An isolated antibody that specifically binds to an epitope, isoform or variant of human heparanase may, however, have cross-reactivity to other related antigens, e.g., from other species (e.g., heparanase species homologs). Moreover, an isolated antibody may be substantially free of other cellular material and/or chemicals. In one embodiment of the invention, a combination of “isolated” monoclonal antibodies having different specificities are combined in a well defined composition.

As used herein, “specific binding” refers to antibody binding to a predetermined antigen. Typically, the antibody binds with an affinity of at least about 1×10⁷ M⁻¹, and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen”.

As used herein, the term “high affinity” for an IgG antibody refers to a binding affinity (K_{A}) of at least about 10⁷ M⁻¹, preferably at least about 10⁸ M^{-1,} more preferably at least about 10⁹M⁻¹, and still more preferably at least about 10¹⁰ M⁻¹, or greater. However, “high affinity” binding can vary for other antibody isotypes. For example, “high affinity” binding for an IgM isotype refers to a binding affinity of at least about 1×10⁷M⁻¹.

The term “K_{assoc}”, as used herein, is intended to refer to the association constant of a particular antibody-antigen interaction.

The term “K_{diss}”, as used herein, is intended to refer to the dissociation constant of a particular antibody-antigen interaction.

The term “K_{A}”, as used herein, is intended to refer to the binding affinity of a particular antibody and is obtained by dividing the K_{assoc} by the K_{diss}.

As used herein, “isotype” refers to the antibody class (e.g., IgM or IgG1) that is encoded by heavy chain constant region genes.

As used herein, “isotype switching” refers to the phenomenon by which the class, or isotype, of an antibody changes from one Ig class to one of the other Ig classes.

As used herein, “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

As used herein, “glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the nonhuman transgenic animal, when one of ordinary skill in the art would recognize the glycosylation pattern of the heterologous antibody as being more similar to said pattern of glycosylation in the species of the nonhuman transgenic animal than to the species from which the CH genes of the transgene were derived.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

The term “nucleic acid molecule”, as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding antibodies or antibody portions (e.g., V_{H}, V_{L}, CDR3) that bind to heparanase, is intended to refer to a nucleic acid molecule in which the nucleotide sequences encoding the antibody or antibody portion are free of other nucleotide sequences encoding antibodies or antibody portions that bind antigens other than heparanase, which other sequences may naturally flank the nucleic acid in human genomic DNA. In one embodiment, the human anti-heparanase antibody, or portion thereof, includes the nucleotide or amino acid sequence of 2H8, 22D9, 13B2, 5G10, or 1E7, as well as heavy chain (V_{H}) and light chain (V_{L}) variable regions having the sequences shown in SEQ ID NOs: 1, 3, 5, 7, 9, 11, 13, 15, 17, or 19, and SEQ ID NOs: 2, 4, 6, 8, 10, 12, 14, 16, 18, or 20, respectively.

As disclosed and claimed herein, the sequences set forth in SEQ ID NOs: 1-20 include “conservative sequence modifications”, i.e., nucleotide and amino acid sequence modifications which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into SEQ ID NOs:1-20 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a human anti-heparanase antibody is preferably replaced with another amino acid residue from the same side chain family.

Alternatively, in another embodiment, mutations can be introduced randomly along all or part of a anti-heparanase antibody coding sequence, such as by saturation mutagenesis, and the resulting modified anti-heparanase antibodies can be screened for binding activity.

Accordingly, antibodies encoded by the (heavy and light chain variable region) nucleotide sequences disclosed herein and/or containing the (heavy and light chain variable region) amino acid sequences disclosed herein (i.e., SEQ ID NOs: 1-20) include substantially similar antibodies encoded by or containing similar sequences which have been conservatively modified. Further discussion as to how such substantially similar antibodies can be generated based on the partial (i.e., heavy and light chain variable regions) sequences disclosed herein as SEQ ID Nos 1-20 is provided below.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid compositions of the present invention, while often in a native sequence (except for modified restriction sites and the like), from either cDNA, genomic or mixtures may be mutated, thereof in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, CHO cells, transfectomas, and lymphocytic cells.

As used herein, the term “subject” includes any human or nonhuman animal. The term “nonhuman animal” includes all vertebrates, e.g., mammals and non-mammals, such as nonhuman primates, sheep, dog, cow, chickens, amphibians, reptiles, etc.

The terms “transgenic, nonhuman animal” refers to a nonhuman animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-heparanase antibodies when immunized with heparanase antigen and/or cells expressing heparanase. The human heavy chain transgene can be integrated into the chromosomal DNA of the mouse, as is the case for transgenic, e.g., HuMAb mice, or the human heavy chain transgene can be maintained extrachromosomally, as is the case for transchromosomal (e.g., KM) mice as described in WO 02/43478. Such transgenic and transchromosomal mice are capable of producing multiple isotypes of human monoclonal antibodies to heparanase (e.g., IgG, IgA and/or IgE) by undergoing V-D-J recombination and isotype switching.

Various aspects of the invention are described in further detail in the following subsections.

I. Production of Human Antibodies to Heparanase

Human monoclonal antibodies of the invention can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes.

The preferred animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known.

In a preferred embodiment, human monoclonal antibodies directed against heparanase can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “transgenic mice.”

The HuMAb mouse contains a human immunoglobulin gene miniloci that encodes unrearranged human heavy (t and y) and K light chain immunoglobulin sequences, together with targeted mutations that inactivate the endogenous μ and κ chain loci (Lonberg, N. et al. (1994) Nature 368 (6474): 856-859). Accordingly, the mice exhibit reduced expression of mouse IgM or κ and in response to immunization, the introduced human heavy and light chain transgenes, undergo class switching and somatic mutation to generate high affinity human IgG κ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. Vol. 13: 65-93, and Harding, F. and Lonberg, N. (1995) Ann. N. Y Acad. Sci 764: 536-546). The preparation of HuMAb mice is described in detail in Taylor, L. et al. (1992) Nucleic Acids Research 20:6287-6295; Chen, J. et al (1993) International Immunology 5: 647-656; Tuaillon et al. (1994) J. Immunol. 152:2912-2920; Lonberg et al., (1994) Nature 368(6474): 856-859; Lonberg, N. (1994) Handbook of Experimental Pharmacology 113:49-101; Taylor, L. et al. (1994) International Immunology 6: 579-591; Lonberg, N. and Huszar, D. (1995) Intern. Rev. Immunol. Vol. 13:65-93; Harding, F. and Lonberg, N. (1995) Ann. N.Y. Acad. Sci 764:536-546; Fishwild, D. et al. (1996) Nature Biotechnology 14:845-851, the contents of all of which are hereby incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay, as well as U.S. Pat. No. 5,545,807 to Surani et al.; International Publication Nos. WO 98/24884, WO 94/25585, WO 93/1227, WO 92/22645, WO 92/03918 and WO 01/09187, the disclosures of all of which are hereby incorporated by reference in their entity.

The KM mouse contains a human heavy chain transchromosome and a human kappa light chain transgene. The endogenous mouse heavy and light chain genes also have been disrupted in the KM mice such that immunization of the mice leads to production of human immunoglobulins rather than mouse immunoglobulins. Construction of KM mice and their use to raise human immunoglobulins is described in detail in International Publication WO 02/43478, the disclosure of which is hereby incorporated by reference herein in its entirety.

Immunizations

To generate fully human monoclonal antibodies to heparanase, transgenic or transchromosomal mice containing human immunoglobulin genes (e.g., HCo12, HCo7 or KM mice) can be immunized with a purified or enriched preparation of heparanase antigen and/or cells expressing heparanase, as described, for example, by Lonberg et al. (1994) Nature 368(6474): 856-859; Fishwild et al. (1996) Nature Biotechnology 14: 845-851 and WO 98/24884. Alternatively, mice can be immunized with DNA encoding human heparanase. Preferably, the mice will be 6-16 weeks of age upon the first infusion. For example, a purified or enriched preparation (5-50 μg) of the heparanase antigen can be used to immunize the HuMAb mice intraperitoneally. In the event that immunizations using a purified or enriched preparation of the heparanase antigen do not result in antibodies, mice can also be immunized with cells expressing heparanase, e.g., a cell line, to promote immune responses.

Cumulative experience with various antigens has shown that the HuMAb transgenic mice respond best when initially immunized intraperitoneally (IP) or subcutaneously (SC) with antigen in complete Freund's adjuvant, followed by every other week IP/SC immunizations (up to a total of 10) with antigen in incomplete Freund's adjuvant. The immune response can be monitored over the course of the immunization protocol with plasma samples being obtained by retroorbital bleeds. The plasma can be screened by ELISA (as described below), and mice with sufficient titers of anti-heparanase human immunoglobulin can be used for fusions. Mice can be boosted intravenously with antigen 3 days before sacrifice and removal of the spleen.

Generation of Hybridomas Producing Human Monoclonal Antibodies to Heparanase

To generate hybridomas producing human monoclonal antibodies to heparanase, splenocytes and lymph node cells from immunized mice can be isolated and fused to an appropriate immortalized cell line, such as a mouse myeloma cell line. The resulting hybridomas can then be screened for the production of antigen-specific antibodies. For example, single cell suspensions of splenic lymphocytes from immunized mice can be fused to SP2/0-Ag8.653 nonsecreting mouse myeloma cells (ATCC, CRL 1580) with 50% PEG (w/v). Cells can be plated at approximately 1×10⁵ in flat bottom microtiter plate, followed by a two week incubation in selective medium containing besides usual reagents 10% fetal Clone Serum, 5-10% origen hybridoma cloning factor (IGEN) and 1X HAT (Sigma). After approximately two weeks, cells can be cultured in medium in which the HAT is replaced with HT. Individual wells can then be screened by ELISA for human anti-heparanase monoclonal IgM and IgG antibodies. Once extensive hybridoma growth occurs, medium can be observed usually after 10-14 days. The antibody secreting hybridomas can be replated, screened again, and if still positive for human IgG, anti-heparanase monoclonal antibodies can be subcloned at least twice by limiting dilution. The stable subclones can then be cultured in vitro to generate antibody in tissue culture medium for characterization.

Generation of Transfectomas Producing Human Monoclonal Antibodies to Heparanase

Human antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the VI, segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or β-globin promoter.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufmnan and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Use of Partial Antibody Sequences to Express Intact Antibodies

Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; and Queen, C. et al. (1989) Proc. Natl. Acad. See. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. Germline gene sequences will also differ from the sequences of a high affinity secondary repertoire antibody at individual evenly across the variable region. For example, somatic mutations are relatively infrequent in the amino-terminal portion of framework region. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody (see WO 99/45962,.which is herein incorporated by referenced for all purposes). Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. For this reason, it is necessary to use the corresponding germline leader sequence for expression constructs. To add missing sequences, cloned cDNA sequences can be combined with synthetic oligonucleotides by ligation or PCR amplification. Alternatively, the entire variable region can be synthesized as a set of short, overlapping, oligonucleotides and combined by PCR amplification to create an entirely synthetic variable region clone. This process has certain advantages such as elimination or inclusion or particular restriction sites, or optimization of particular codons.

The nucleotide sequences of heavy and light chain transcripts from hybridomas are used to design an overlapping set of synthetic oligonucleotides to create synthetic V sequences with identical amino acid coding capacities as the natural sequences. The synthetic heavy and kappa chain sequences can differ from the natural sequences in three ways: strings of repeated nucleotide bases are interrupted to facilitate oligonucleotide synthesis and PCR amplification; optimal translation initiation sites are incorporated according to Kozak's rules (Kozak (1991) J. Biol. Chem. 266:19867-19870); and HindIII sites are engineered upstream of the translation initiation sites.

For both the heavy and light chain variable regions, the optimized coding, and corresponding non-coding, strand sequences are broken down into 30-50 nucleotide approximately the midpoint of the corresponding non-coding oligonucleotide. Thus, for each chain, the oligonucleotides can be assembled into overlapping double stranded sets that span segments of 150-400 nucleotides. The pools are then used as templates to produce PCR amplification products of 150-400 nucleotides. Typically, a single variable region oligonucleotide set will be broken down into two pools which are separately amplified to generate two overlapping PCR products. These overlapping products are then combined by PCR amplification to form the complete variable region. It may also be desirable to include an overlapping fragment of the heavy or light chain constant region (including the BbsI site of the kappa light chain, or the AgeI site of the gamma heavy chain) in the PCR amplification to generate fragments that can easily be cloned into the expression vector constructs.

The reconstructed heavy and light chain variable regions are then combined with cloned promoter, translation initiation, constant region, 3′ untranslated, polyadenylation, and transcription termination, sequences to form expression vector constructs. The heavy and light chain expression constructs can be combined into a single vector, co-transfected, serially transfected, or separately transfected into host cells which are then fused to form a host cell expressing both chains.

Plasmids for use in construction of expression vectors for human IgGκ are described below. The plasmids were constructed so that PCR amplified V heavy and V kappa light chain cDNA sequences could be used to reconstruct complete heavy and light chain minigenes. These plasmids can be used to express completely human, or chimeric IgG₁κ or IgG₄κ antibodies. Similar plasmids can be constructed for expression of other heavy chain isotypes, or for expression of antibodies comprising lambda light chains.

Thus, in another aspect of the invention, the structural features of an human anti-heparanase antibodies of the invention, e.g., 2H8, 22D9, 13B2, 5G10, or 1E7, are used to create structurally related human anti-heparanase antibodies that retain at least one functional property of the antibodies of the invention, such as binding to heparanase. More specifically, one or more CDR regions of 2H8, 22D9, 13B2, 5G10, or 1E7 can be combined recombinantly with known human framework regions and CDRs to create additional, recombinantly-engineered, human anti-heparanase antibodies of the invention.

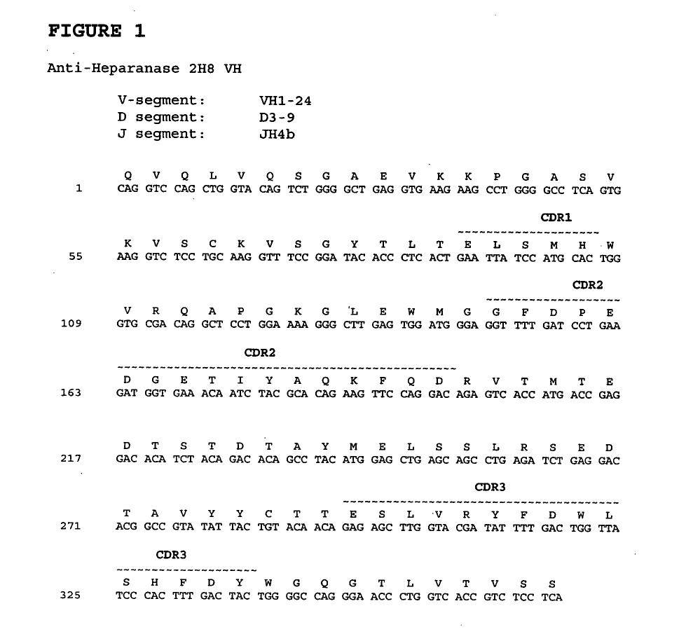

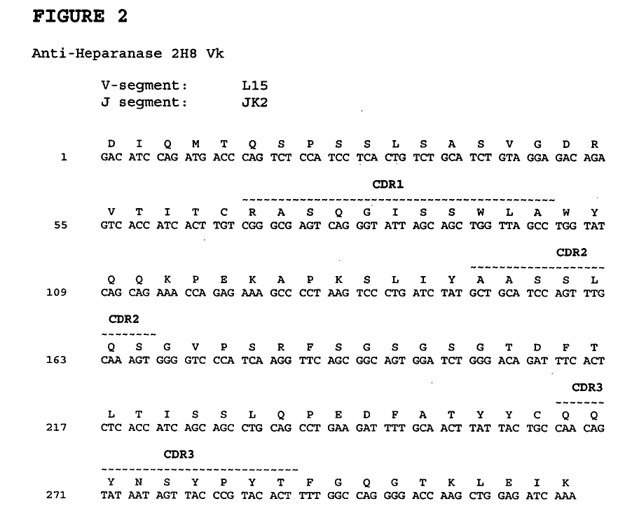

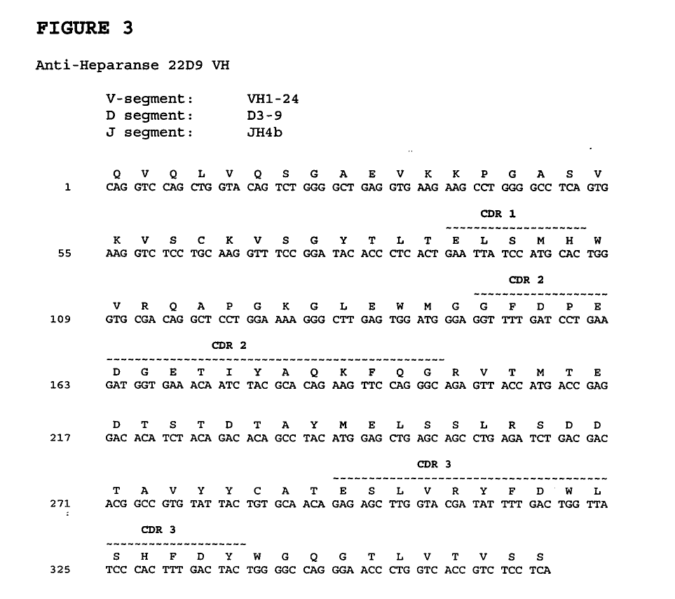

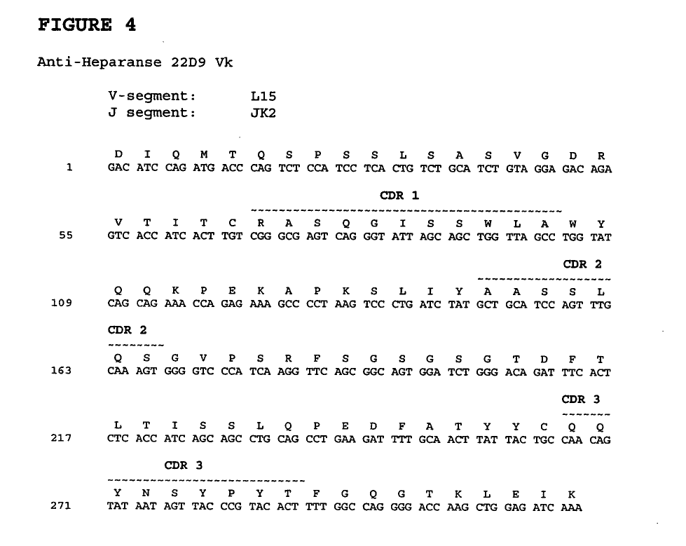

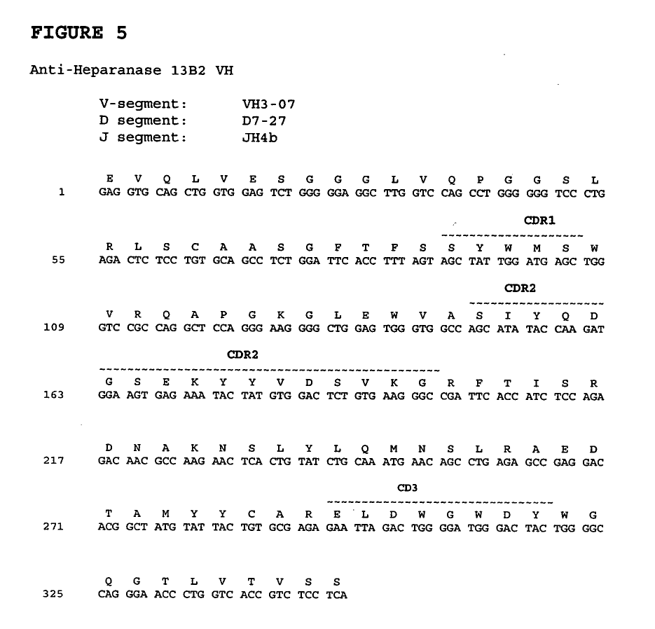

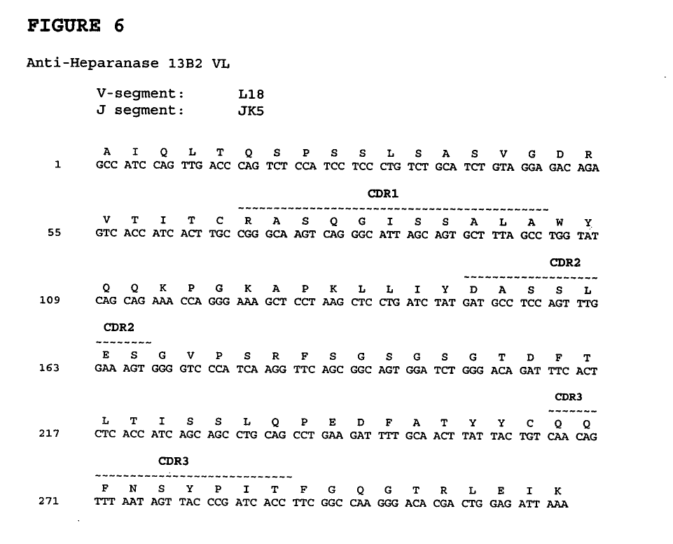

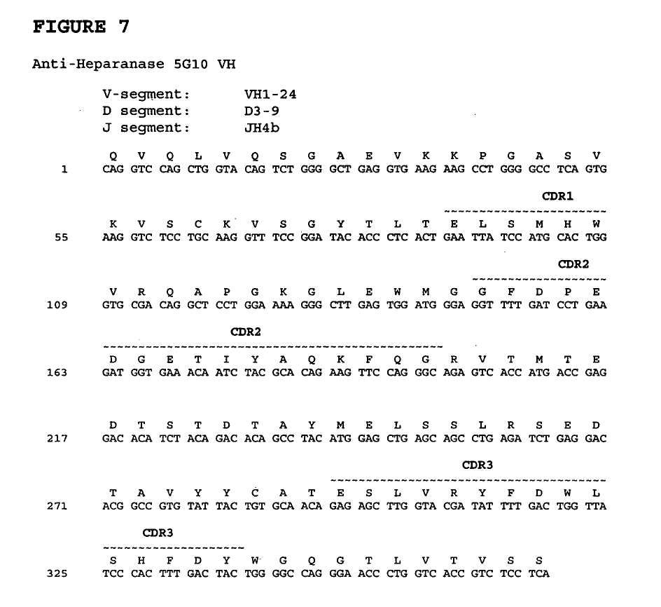

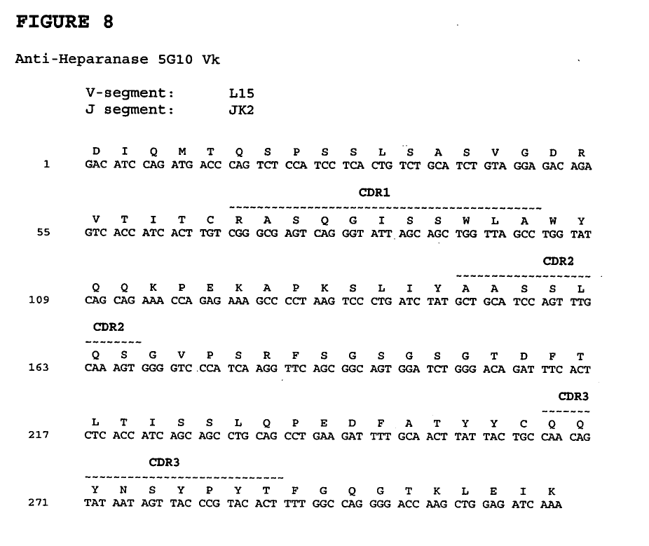

Accordingly, in another embodiment, the invention provides a method for preparing an anti-heparanase antibody comprising: preparing an antibody comprising (1) human heavy chain framework regions and human heavy chain CDRs, wherein at least one of the human heavy chain CDRs comprises an amino acid sequence selected from the amino acid sequences of CDRs shown in FIGS. 1, 3, 5, 7, or 9 (or corresponding amino acid residues in SEQ ID NOs: 2, 6, 10, 14, or 18); and (2) human light chain framework regions and human light chain CDRs, wherein at least one of the human light chain CDRs comprises an amino acid sequence selected from the amino acid sequences of CDRs shown in FIGS. 2, 4, 6, 8, or 10 (or corresponding amino acid residues in SEQ ID NOs: 4, 8, 12, 16, or 20); wherein the antibody retains the ability to bind to heparanase.

The ability of the antibody to bind heparanase can be determined using standard binding assays, such as those set forth in the Examples (e.g., an ELISA). Since it is well known in the art that antibody heavy and light chain CDR3 domains play a particularly important role in the binding specificity/affinity of an antibody for an antigen, the recombinant antibodies of the invention prepared as set forth above preferably comprise the heavy and light chain CDR3s of 2H8, 22D9, 12B2, 5G10, or 1E7. The antibodies further can comprise the CDR2s of 2H8, 22D9, 12B2, 5G10, or 1E7. The antibodies further can comprise the CDR1s of 2H8, 22D9, 12B2, 5G10, or 1E7. Accordingly, the invention further provides anti-heparanase antibodies comprising: (1) human heavy chain framework regions, a human heavy chain CDR1 region, a human heavy chain CDR2 region, and a human heavy chain CDR3 region, wherein the human heavy chain CDR3 region is the CDR3 of 2H8, 22D9, 12B2, 5G10, or 1E7 as shown in FIGS. 1, 3, 5, 7, or 9 (or corresponding amino acid residues in SEQ ID NOs: 2, 6, 10, 14, or 18); and (2) human light chain framework regions, a human light chain CDR1 region, a human light chain CDR2 region, and a human light chain CDR3 region, wherein the human light chain CDR3 region is the CDR3 of 2H8, 22D9, 12B2, 5G10, or 1E7 as shown in FIGS. 2, 4, 6, 8, or 10 (or corresponding amino acid residues in SEQ ID NO: 4, 8, 12, 16, or 20), wherein the antibody binds heparanase. The antibody may further comprise the heavy chain CDR2 and/or the light chain CDR2 of 2H8, 22D9, 12B2, 5G10, or 1E7. The antibody may further comprise the heavy chain CDR1 and/or the light chain CDR1 of 2H8, 22D9, 12B2, 5G10, or 1E7.

Preferably, the CDR1, 2, and/or 3 of the engineered antibodies described above comprise the exact amino acid sequence(s) as those of 2H8, 22D9, 12B2, 5G10, or 1E7 disclosed herein. However, the ordinarily skilled artisan will appreciate that some deviation from the exact CDR sequences of 2H8, 22D9, 12B2, 5G10, or 1E7 may be possible while still retaining the ability of the antibody to bind heparanase effectively (e.g., conservative substitutions). Accordingly, in another embodiment, the engineered antibody may be composed of one or more CDRs that are, for example, at least 90%, 95%, 98% or 99.5% identical to one or more CDRs of 2H8, 22D9, 12B2, 5G10, or 1E7.

In addition or alternatively to simply binding heparanase, engineered antibodies such as those described above may be selected for their retention of other functional properties of antibodies of the invention, such as:

(1) high affinity binding to heparanase;

(2) binding to a unique epitope on heparanase (to eliminate the possibility that monoclonal antibodies with complimentary activities when used in combination would compete for binding to the same epitope);

(3) inhibition of human heparanase activity; and/or (4) inhibition of cell growth and/or migration induced by human heparanase.

Characterization of Binding of Human Monoclonal Antibodies to Heparanase Human monoclonal antibodies of the invention can be tested for binding to heparanase by, for example, standard ELISA. Briefly, microtiter plates are coated with purified heparanase at 0.25 μg/ml in PBS, and then blocked with 5% bovine serum albumin in PBS. Dilutions of plasma from heparanase-immunized mice are added to each well and incubated for 1-2 hours at 37° C. The plates are washed with PBS/Tween and then incubated with a goat-anti-human IgG Fc-specific polyclonal reagent conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with heparanase immunogen. Hybridomas that bind with high avidity to heparanase will be subcloned and further characterized. One clone from each hybridoma, which retains the reactivity of the parent cells (by ELISA), can be chosen for making a 5-10 vial cell bank stored at −140° C., and for antibody purification.

To purify human anti-heparanase antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected human anti-heparanase monoclonal antibodies bind to unique epitopes, each antibody can be biotinylated using commercially available reagents (Pierce, Rockford, Ill.). Competition studies using unlabeled monoclonal antibodies and biotinylated Monoclonal antibodies can be performed using heparanase coated-ELISA plates as described above. Biotinylated MAb binding can be detected with a strep-avidin-alkaline phosphatase probe.

To determine the isotype of purified antibodies, isotype ELISAs can be performed. Wells of microtiter plates can be coated with 10 μg/ml of anti-human Ig overnight at 4° C. After blocking with 5% BSA, the plates are reacted with 10 pg/ml of monoclonal antibodies or purified isotype controls, at ambient temperature for 2 hours. The wells can then be reacted with either human IgG isotype or human IgM-specific alkaline phosphatase-conjugated probes. Plates are developed and analyzed as described above.

In order to demonstrate binding of monoclonal antibodies to live cells expressing the heparanase, flow cytometry can be used. Briefly, cell lines expressing heparanase (grown under standard growth conditions) are mixed with various concentrations of monoclonal antibodies in PBS containing 0.1% Tween 80 and 20% mouse serum, and incubated at 37° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Anti-heparanase human IgGs can be further tested for reactivity with heparanase antigen by Western blotting. Briefly, cell extracts from cells expressing heparanase can be prepared and subjected to sodium dodecyl sulfate (SDS) polyacrylamide gel electrophoresis. After electrophoresis, the separated antigens will be transferred to nitrocellulose membranes, blocked with 20% mouse serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Inhibitory Activities of Human Monoclonal Antibodies to Heparanase

In addition to binding, human monoclonal anti-heparanase antibodies can be tested for their ability to inhibit heparanase activity as measured by a variety of cellular assays. The testing of monoclonal antibody activity in vitro will provide an initial screening prior to testing in vivo models. For example, measurements of heparanase activities can be carried out as described previously (Vlodavsky, I. et al. (1999) Mammalian heparanase: gene cloning, expression and function in tumor progression and metastasis. Nature Med. 5: 793-802) using ³⁵S labeled heparin sulphate. Inhibition of heparanase activity can also be determined using standard ELISA protocols.

The inhibitory effect of anti-heparanase antibodies on heparanase-induced cell motility, e.g., tumor cell motility, can be measured with an in vitro motility/migration assay utilizing PC3 cells (a prostate derived cell line) and standard procedures. Briefly, chamber inserts (with 8 μM pore size) are placed into wells containing the appropriate media. PC3 cells in logarithmic growth phase are plated in the upper chamber at a density of about 1×10⁵ cells per well in a volume of about 180 μl with or without monoclonal antibodies and controls (20 μl). Cells that migrate through to the bottom chamber can be counted and cell motility determined.

Other methods for measuring the inhibitory effects of anti-heparanase antibodies include contacting such antibodies to a tumor cell line, e.g., MDA-MB 435 cells, or administering the antibodies to a subject suffering from a disorder mediated by heparanase, e.g., breast cancer, and calculating the decrease in tumor cell volumes as a measurement of tumor cell inhibition. A preferred system for examining inhibition of cell growth using the breast tumor cell line MDA-MB 435 is described in Example 5.

Human monoclonal antibodies of the present invention can also be tested for their ability to inhibit other cellular activities induced by human heparanase, such as inflammatory or autoimmune responses.

Preferred human monoclonal antibodies-of the invention are capable of inhibiting at least one activity of human heparanase. In a particular embodiment, the human monoclonal antibodies are used in combination, e.g., as a pharmaceutical composition comprising two or more anti-heparanase monoclonal antibodies or fragments thereof. For example, human anti-heparanase monoclonal antibodies having different, but complementary activities can be combined in a single therapy to achieve a desired therapeutic or diagnostic effect. An illustration of this would be a composition containing an anti-heparanase human monoclonal antibody that mediates highly effective killing of target cells in the presence of effector cells, combined with another human anti-heparanase monoclonal antibody that inhibits the growth of cells expressing heparanase.

II. Production of Transgenic Non-human Animals Which Generate Human Monoclonal Anti-Heparanase Antibodies

In yet another aspect, the invention provides transgenic and transchromosomal nonhuman animals, such as transgenic or transchromosomal mice, which are capable of expressing human monoclonal antibodies that specifically bind to heparanase. In a particular embodiment, the invention provides a transgenic or transchromosomal mouse having a genome comprising a human heavy chain transgene, such that the mouse produces human anti-heparanase antibodies when immunized with heparanase antigen and/or cells expressing heparanase. The human heavy chain transgene can be integrated into the chromosomal DNA of the mouse, as is the case for transgenic, e.g., HuMAb mice, as described in detail herein and exemplified. Alternatively, the human heavy chain transgene can be maintained extrachromosomally, as is the case for transchromosomal (e.g., KM) mice as described in WO 02/43478. Such transgenic and transchromosomal animals are capable of producing multiple isotypes of human monoclonal antibodies to heparanase (e.g., IgG, IgA and/or IgE) by undergoing V-D-J recombination and isotype switching. Isotype switching may occur by, e.g., classical or non-classical isotype switching.

The design of a transgenic or transchromosomal nonhuman animal that responds to foreign antigen stimulation with a heterologous antibody repertoire, requires that the heterologous immunoglobulin transgenes contained within the transgenic animal function correctly throughout the pathway of B-cell development. This includes, for example, isotype switching of the heterologous heavy chain transgene. Accordingly, transgenes are constructed so as to produce isotype switching and one or more of the following of antibodies: (1) high level and cell-type specific expression, (2) functional gene rearrangement, (3) activation of and response to allelic exclusion, (4) expression of a sufficient primary repertoire, (5) signal transduction, (6) somatic hypermutation, and (7) domination of the transgene antibody locus during the immune response.

Not all of the foregoing criteria need be met. For example, in those embodiments wherein the endogenous immunoglobulin loci of the transgenic animal are functionally disrupted, the transgene need not activate allelic exclusion. Further, in those embodiments wherein the transgene comprises a functionally rearranged heavy and/or light chain immunoglobulin gene, the second criteria of functional gene rearrangement is unnecessary, at least for that transgene which is already rearranged. For background on molecular immunology, see, Fundamental Immunology, 2nd edition (1989), Paul William E., ed. Raven Press, N.Y.

In certain embodiments, the transgenic or transchromosomal nonhuman animals used to generate the human monoclonal antibodies of the invention contain rearranged, unrearranged or a combination of rearranged and unrearranged heterologous immunoglobulin heavy and light chain transgenes in the germline of the transgenic animal. Each of the heavy chain transgenes comprises at least one C_{H} gene. In addition, the heavy chain transgene may contain functional isotype switch sequences, which are capable of supporting isotype switching of a heterologous transgene encoding multiple C_{H} genes in the B-cells of the transgenic animal. Such switch sequences may be those which occur naturally in the germline immunoglobulin locus from the species that serves as the source of the transgene C_{H} genes, or such switch sequences may be derived from those which occur in the species that is to receive the transgene construct (the transgenic animal). For example, a human transgene construct that is used to produce a transgenic mouse may produce a higher frequency of isotype switching events if it incorporates switch sequences similar to those that occur naturally in the mouse heavy chain locus, as presumably the mouse switch sequences are optimized to function with the mouse switch recombinase enzyme system, whereas the human switch sequences are not. Switch sequences may be isolated and cloned by conventional cloning methods, or may be synthesized de novo from overlapping synthetic oligonucleotides designed on the basis of published sequence information relating to immunoglobulin switch region sequences (Mills et al., Nucl. Acids Res. 15:7305-7316 (1991); Sideras et al., Intl. Immunol. 1:631-642 (1989)). For each of the foregoing transgenic animals, functionally rearranged heterologous heavy and light chain immunoglobulin transgenes are found in a significant fraction of the B-cells of the transgenic animal (at least 10 percent).

The transgenes used to generate the transgenic nonhuman animals used to produce the human monoclonal antibodies of the invention include a heavy chain transgene comprising DNA encoding at least one variable gene segment, one diversity gene segment, one joining gene segment and at least one constant region gene segment. The immunoglobulin light chain transgene comprises DNA encoding at least one variable gene segment, one joining gene segment and at least one constant region gene segment. The gene segments encoding the light and heavy chain gene segments are heterologous to the transgenic animal in that they are derived from, or correspond to, DNA encoding immunoglobulin heavy and light chain gene segments from a species not consisting of the transgenic nonhuman animal. In one aspect of the invention, the transgene is constructed such that the individual gene segments are unrearranged, i.e., not rearranged so as to encode a functional immunoglobulin light or heavy chain. Such unrearranged transgenes support recombination of the V, D, and J gene segments (functional rearrangement) and preferably support incorporation of all or a portion of a D region gene segment in the resultant rearranged immunoglobulin heavy chain within the transgenic animal when exposed to heparanase antigen.

In an alternate embodiment, the transgenes comprise an unrearranged “~mini-locus” v. Such transgenes typically comprise a substantial portion of the C, D, and J segments as well as a subset of the V gene segments. In such transgene constructs, the various regulatory sequences, e.g., promoters, enhancers, class switch regions, splice-donor and splice-acceptor sequences for RNA processing, recombination signals and the like, comprise corresponding sequences derived from the heterologous DNA. Such regulatory sequences may be incorporated into the transgene from the same or a related species of the nonhuman animal used in the invention. For example, human immunoglobulin gene segments may be combined in a transgene with a rodent immunoglobulin enhancer sequence for use in a transgenic mouse. Alternatively, synthetic regulatory sequences may be incorporated into the transgene, wherein such synthetic regulatory sequences are not homologous to a functional DNA sequence that is known to occur naturally in the genomes of mammals. Synthetic regulatory sequences are designed according to consensus rules, such as, for example, those specifying the permissible sequences of a splice-acceptor site or a promoter/enhancer motif. For example, a minilocus comprises a portion of the genomic immunoglobulin locus having at least one internal (i.e., not at a terminus of the portion) deletion of a non-essential DNA portion (e.g., intervening sequence; intron or portion thereof) as compared to the naturally-occurring germline Ig locus.

In a preferred embodiment of the invention, the transgenic or transchromosomal animal used to generate human antibodies to heparanase contains at least one, typically 2-10, and sometimes 25-50 or more copies of the transgene described in Example 12 of WO 98/24884 (e.g., pHC1 or pHC2) bred with an animal containing a single copy of a light chain transgene described in Examples 5, 6, 8, or 14 of WO 98/24884, and the offspring bred with the J_{H} deleted animal described in Example 10 of WO 98/24884. Animals are bred to homozygosity for each of these three traits. Such animals have the following genotype: a single copy (per haploid set of chromosomes) of a human heavy chain unrearranged mini-locus (described in Example 12 of WO 98/24884), a single copy (per haploid set of chromosomes) of a rearranged human K light chain construct (described in Example 14 of WO 98/24884), and a deletion at each endogenous mouse heavy chain locus that removes all of the functional J_{H} segments (described in Example 10 of WO 98/24884). Such animals are bred with mice that are homozygous for the deletion of the J_{H} segments (Example 10 of WO 9$8/24884) to produce offspring that are homozygous for the J_{H} deletion and hemizygous for the human heavy and light chain constructs. The resultant animals are injected with antigens and used for production of human monoclonal antibodies against these antigens.

B cells isolated from such an animal are monospecific with regard to the human heavy and light chains because they contain only a single copy of each gene. Furthermore, they will be monospecific with regards to human or mouse heavy chains because both endogenous mouse heavy chain gene copies are nonfunctional by virtue of the deletion spanning the J_{H} region introduced as described in Examples 9 and 12 of WO 98/24884. Furthermore, a substantial fraction of the B cells will be monospecific with regards to the human or mouse light chains because expression of the single copy of the rearranged human κ light chain gene will allelically and isotypically exclude the rearrangement of the endogenous mouse κ and lambda chain genes in a significant fraction of B-cells.

Preferred transgenic and transchromosomal nonhuman animals, e.g., mice, will exhibit immunoglobulin production with a significant repertoire, ideally substantially similar to that of a native mouse. Thus, for example, in embodiments where the endogenous Ig genes have been inactivated, the total immunoglobulin levels will range from about 0.1 to 10 mg/ml of serum, preferably 0.5 to 5 mg/ml, ideally at least about 1.0 mg/ml. When a transgene capable of effecting a switch to IgG from IgM has been introduced into the transgenic mouse, the adult mouse ratio of serum IgG to IgM is preferably about 10:1. The IgG to IgM ratio will be much lower in the immature mouse. In general, greater than about 10%, preferably 40 to 80% of the spleen and lymph node B cells express exclusively human IgG protein.

The repertoire will ideally approximate that shown in a native mouse, usually at least about 10% as high, preferably 25 to 50% or more. Generally, at least about a thousand different immunoglobulins (ideally IgG), preferably 10⁴ to 10⁶ or more, will be produced, depending primarily on the number of different V, J and D regions introduced into the mouse genome. These immunoglobulins will typically recognize about one-half or more of highly antigenic proteins, e.g., staphylococcus protein A. Typically, the immunoglobulins will exhibit an affinity (K_{D}) for preselected antigens of below 10⁻⁷ M, such as of below 10⁻⁸ M, 10⁻⁹ M or. 10⁻¹⁰ M or even lower.

In some embodiments, it may be preferable to generate nonhuman animals with predetermined repertoires to limit the selection of V genes represented in the antibody response to a predetermined antigen type. A heavy chain transgene having a predetermined repertoire may comprise, for example, human V_{H} genes which are preferentially used in antibody responses to the predetermined antigen type in humans. Alternatively, some V_{H} genes may be excluded from a defined repertoire for various reasons (e.g., have a low likelihood of encoding high affinity V regions for the predetermined antigen; have a low propensity to undergo somatic mutation and affinity sharpening; or are immunogenic to certain humans). Thus, prior to rearrangement of a transgene containing various heavy or light chain gene segments, such gene segments may be readily identified, e.g., by hybridization or DNA sequencing, as being from a species of organism other than the transgenic animal.

Transgenic and transchromosomal nonhuman animals, e.g., mice, as described above can be immunized with, for example, a purified or recombinant preparation of heparanase antigen and/or cells expressing heparanase. Alternatively, the transgenic animals can be immunized with DNA encoding human heparanase. The animals will then produce B cells which undergo class-switching via intratransgene switch recombination (cis-switching) and express immunoglobulins reactive with heparanase. The immunoglobulins can be human antibodies (also referred to as “human sequence antibodies”), wherein the heavy and light chain polypeptides are encoded by human transgene sequences, which may include sequences derived by somatic mutation and V region recombinatorial joints, as well as germline-encoded sequences; these human antibodies can be referred to as being substantially identical to a polypeptide sequence encoded by a human V_{L} or V_{H} gene segment and a human J_{L} or D_{H} and J_{H} segment, even though other non-germline sequences may be present as a result of somatic mutation and differential V-J and V-D-J recombination joints. The variable regions of each antibody chain are typically at least 80 percent encoded by human germline V, J, and, in the case of heavy chains, D, gene segments; frequently at least 85 percent of the variable regions are encoded by human germline sequences present on the transgene; of ten 90 or 95 percent or more of the variable region sequences are encoded by human germline sequences present on the transgene. However, since non-germline sequences are introduced by somatic mutation and VJ and VDJ joining, the human sequence antibodies will frequently have some variable region sequences (and less frequently constant region sequences) which are not encoded by human V, D, or J gene segments as found in the human transgene(s) in the germline of the mice. Typically, such non-germline sequences (or individual nucleotide positions) will cluster in or near CDRs, or in regions where somatic mutations are known to cluster.

Human antibodies which bind to the predetermined antigen can result from isotype switching, such that human antibodies comprising a human sequence γ chain (such as γ1, γ2a, γ2B, or γ3) and a human sequence light chain (such as kappa) are produced. Such isotype-switched human antibodies of ten contain one or more somatic mutation(s), typically in the variable region and of ten in or within about 10 residues of a CDR) as a result of affinity maturation and selection of B cells by antigen, particularly subsequent to secondary (or subsequent) antigen challenge. These high affinity human antibodies may have binding affinities (K_{D}) of below 10⁻⁷ M, such as of below 10⁻⁸ M, 10⁻⁹ M or 10⁻¹⁰ M or even lower.

Another aspect of the invention includes B cells derived from transgenic or transchromosomal nonhuman animals as described herein. The B cells can be used to generate hybridomas expressing human monoclonal antibodies which bind with high affinity (e.g., lower than 10⁻⁷ M) to human heparanase. Thus, in another embodiment, the invention provides a hybridoma which produces a human antibody having an affinity (K_{D}) of below 10⁻⁷ M, such as of below 10⁻⁸ M, 10⁻⁹ M or 10⁻¹⁰ M or even lower when determined by surface plasmon resonance (SPR) technology in a BIACORE 3000 instrument using recombinant human heparanase as the analyte and the antibody as the ligand for binding human heparanase, wherein the antibody comprises:

a human sequence light chain composed of (1) a light chain variable region having a polypeptide sequence which is substantially identical to a polypeptide sequence encoded by a human V_{L} gene segment and a human J_{L} segment, and (2) a light chain constant region having a polypeptide sequence which is substantially identical to a polypeptide sequence encoded by a human C_{L} gene segment; and

a human sequence heavy chain composed of a (1) a heavy chain variable region having a polypeptide sequence which is substantially identical to a polypeptide sequence encoded by a human V_{H} gene segment, optionally a D region, and a human J_{H} segment, and (2) a constant region having a polypeptide sequence which is substantially identical to a polypeptide sequence encoded by a human C_{H} gene segment.

The development of high affinity human monoclonal antibodies against heparanase can be facilitated by a method for expanding the repertoire of human variable region gene segments in a transgenic nonhuman animal having a genome comprising an integrated human immunoglobulin transgene, said method comprising introducing into the genome a V gene transgene comprising V region gene segments which are not present in said integrated human immunoglobulin transgene. Often, the V region transgene is a yeast artificial chromosome comprising a portion of a human V_{H} or V_{L} (V_{K}) gene segment array, as may naturally occur in a human genome or as may be spliced together separately by recombinant methods, which may include out-of -order or omitted V gene segments. Often at least five or more functional V gene segments are contained on the YAC. In this variation, it is possible to make a transgenic animal produced by the V repertoire expansion method, wherein the animal expresses an immunoglobulin chain comprising a variable region sequence encoded by a V region gene segment present on the V region transgene and a C region encoded on the human Ig transgene. By means of the V repertoire expansion method, transgenic animals having at least 5 distinct V genes can be generated; as can animals containing at least about 24 V genes or more. Some V gene segments may be non-functional (e.g., pseudogenes and the like); these segments may be retained or may be selectively deleted by recombinant methods available to the skilled artisan, if desired.

Once the mouse germline has been engineered to contain a functional YAC having an expanded V segment repertoire, substantially not present in the human Ig transgene containing the J and C gene segments, the trait can be propagated and bred into other genetic backgrounds, including backgrounds where the functional YAC having an expanded V segment repertoire is bred into a nonhuman animal germline having a different human Ig transgene. Multiple functional YACs having an expanded V segment repertoire may be bred into a germline to work with a human Ig transgene (or multiple human Ig transgenes). Although referred to herein as YAC transgenes, such transgenes when integrated into the genome may substantially lack yeast sequences, such as sequences required for autonomous replication in yeast; such sequences may optionally be removed by genetic engineering (e.g., restriction digestion and pulsed-field gel electrophoresis or other suitable method) after replication in yeast is no longer necessary (i.e., prior to introduction into a mouse ES cell or mouse prozygote). Methods of propagating the trait of human sequence immunoglobulin expression, include breeding a transgenic animal having the human Ig transgene(s), and optionally also having a functional YAC having an expanded V segment repertoire. Both V_{H} and V_{L} gene segments may be present on the YAC. The transgenic animal may be bred into any background desired by the practitioner, including backgrounds harboring other human transgenes, including human Ig transgenes and/or transgenes encoding other human lymphocyte proteins. The invention also provides a high affinity human sequence immunoglobulin produced by a transgenic mouse having an expanded V region repertoire YAC transgene. Although the foregoing describes a preferred embodiment of the transgenic animal used to produce the human monoclonal antibodies of the invention, other embodiments are contemplated which have been classified in four categories:

I. Transgenic animals containing an unrearranged heavy and rearranged light immunoglobulin transgene;

II. Transgenic animals containing an unrearranged heavy and unrearranged light immunoglobulin transgene;

III. Transgenic animals containing a rearranged heavy and an unrearranged light immunoglobulin transgene; and

IV. Transgenic animals containing a rearranged heavy and rearranged light immunoglobulin transgene.

Of these categories of transgenic animal, the order of preference is as follows II>I>III>IV where the endogenous light chain genes (or at least the K gene) have been knocked out by homologous recombination (or other method) and I>II>III>IV where the endogenous light chain genes have not been knocked out and must be dominated by allelic exclusion.

III. Human Antibody Derivatives

In yet another embodiment of the invention, human monoclonal antibodies to heparanase, or antigen-binding portions thereof, can be derivatized or linked to another functional molecule, e.g., another peptide or protein (e.g., a Fab′ fragment) to generate a bispecific or multispecific molecule which binds to multiple binding sites or target epitopes. For example, an antibody or antigen-binding portion of the invention can be functionally linked (e.g., by chemical coupling, genetic fusion, noncovalent association or otherwise) to one or more other binding molecules, such as another antibody, antibody fragment, peptide or binding mimetic.

Accordingly, the present invention includes bispecific and multispecific molecules comprising at least one first binding specificity for heparanase and a second binding specificity for a second target epitope. In a particular embodiment of the invention, the second target epitope is an Fc receptor, e.g., human FcγRI (CD64) or a human Fcα receptor (CD89). Therefore, the invention includes bispecific and multispecific molecules capable of binding both to FcγR, FcαR or FcεR expressing effector cells (e.g., monocytes, macrophages or polymorphonuclear cells (PMNs)), and to target cells expressing heparanase. These bispecific and multispecific molecules target heparanase expressing cells to effector cell and, like the human monoclonal antibodies of the invention, trigger Fc receptor-mediated effector cell activities, such as phagocytosis of a heparanase expressing cells, antibody dependent cell-mediated cytotoxicity (ADCC), cytokine release, or generation of superoxide anion.

Bispecific and multispecific molecules of the invention can further include a third binding specificity, in addition to an anti-Fc binding specificity and an anti-heparanase binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the F_{c} receptor or target cell antigen. The “anti-enhancement factor portion” can bind an F_{c} receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-1 or other immune cell that results in an increased immune response against the target cell).

In one embodiment, the bispecific and multispecific molecules of the invention comprise as a binding specificity at least one antibody, or an antibody fragment thereof, including, e.g., a Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, issued Aug. 7, 1990, the contents of which is expressly incorporated by reference.

In one embodiment bispecific and multispecific molecules of the invention comprise a binding specificity for an FcγR or an FcαR present on the surface of an effector cell, and a second binding specificity for a target cell antigen, e.g., heparanase.

In one embodiment, the binding specificity for an Fc receptor is provided by a human monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII(CD32), and FcγRIII (CD16). In one preferred embodiment, the Fcγ receptor a human high affinity FcγRI. The human FcγRI is a 72 kDa molecule, which shows high affinity for monomeric IgG (10⁸-10⁹ M⁻¹).

The production and characterization of these preferred monoclonal antibodies are described by Fanger et al. in International application WO 88/00052 and in U.S. Pat. No. 4,954,617, the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are MAb 22, MAb 32, MAb 44, MAb 62 and MAb 197. The hybridoma producing MAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol 155 (10): 4996-5002 and PCT/US93/10384. The H22 antibody producing cell line was deposited at the American Type Culture Collection under the designation HA022CL1 and has the accession no. CRL 11177.

In still other preferred embodiments, the binding specificity for an Fc receptor is provided by an antibody that binds to a human IgA receptor, e.g., an Fc-alpha receptor (FcαRI (CD89)), the binding of which is preferably not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al., 1992, J. Immunol. 148:1764).

FcαRI and FcγRI are preferred trigger receptors for use in the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

In other embodiments, bispecific and multispecific molecules of the invention further comprise a binding specificity which recognizes, e.g., binds to, a target cell antigen, e.g., heparanase. In a preferred embodiment, the binding specificity is provided by a human monoclonal antibody of the present invention.

An “effector cell specific antibody” as used herein refers to an antibody or functional antibody fragment that binds the Fc receptor of effector cells. Preferred antibodies for use in the subject invention bind the Fc receptor of effector cells at a site which is not bound by endogenous immunoglobulin.

As used herein, the term “effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Some effector cells express specific Fc receptors and carry out specific immune functions. In preferred embodiments, an effector cell is capable of inducing antibody-dependent cell-mediated cytotoxicity (ADCC), e.g., a neutrophil capable of inducing ADCC. For example, monocytes, macrophages, which express FcR are involved in specific killing of target cells and presenting antigens to other components of the immune system, or binding to cells that present antigens. In other embodiments, an effector cell can phagocytose a target antigen, target cell, or microorganism. The expression of a particular FcR on an effector cell can be regulated by humoral factors such as cytokines. For example, expression of FcγRI has been found to be up-regulated by interferon gamma (IFN-γ). This enhanced expression increases the cytotoxic activity of FcγRI-bearing cells against targets. An effector cell can phagocytose or lyse a target antigen or a target cell.

“Target cell” shall mean any undesirable cell in a subject (e.g., a human or animal) that can be targeted by a composition (e.g., a human monoclonal antibody, a bispecific or a multispecific molecule) of the invention. In preferred embodiments, the target cell is a cell expressing or overexpressing heparanase. Cells expressing heparanase typically include tumor cells, such as melanoma cells, lymphoma cells, prostate carcinoma cells, pancreatic carcinoma cells, bladder carcinoma cells, mast cells, fibrosarcoma cells, rhabdomyosarcoma cells, mastocytoma cells, mammary adenocarcinoma cells, leukemia cells, and rheumatoid fibroblasts. Other cells that can be targeted include lymphocytes, neutrophils, macrophages, eosinophils, T-cells, endothelial cells, and platelets.

While human monoclonal antibodies are preferred, other antibodies which can be employed in the bispecific or multispecific molecules of the invention are murine, chimeric and humanized monoclonal antibodies.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Application WO 87/02671; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

All of the CDRs of a particular human antibody may be replaced with at least a portion of a nonhuman CDR or only some of the CDRs may be replaced with nonhuman CDRs. It is only necessary to replace the number of CDRs required for binding of the humanized antibody to the Fc receptor.

An antibody can be humanized by any method, which is capable of replacing at least a portion of a CDR of a human antibody with a CDR derived from a nonhuman antibody. Winter describes a method which may be used to prepare the humanized antibodies of the present invention (UK Patent Application GB 2188638A), the contents of which is expressly incorporated by reference. The human CDRs may be replaced with nonhuman CDRs using oligonucleotide site-directed mutagenesis as described in International Application WO 94/10332 entitled, Humanized Antibodies to Fc Receptors for Immunoglobulin G on Human Mononuclear Phagocytes.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or substituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the bispecific and multispecific molecule has at least one antigen binding region specific for an FcγR and triggers at least one effector function.

Bispecific and multispecific molecules of the present invention can be made using chemical techniques (see e.g., D. M. Kranz et al. (1981) Proc. Natl. Acad. Sci. USA 78:5807), “polydoma” techniques (See U.S. Pat. No. 4,474,893, to Reading), or recombinant DNA techniques.

In particular, bispecific and multispecific molecules of the present invention can be prepared by conjugating the constituent binding specificities, e.g., the anti-FcR and anti-heparanase binding specificities, using methods known in the art and described in the examples provided herein. For example, each binding specificity of the bispecific and multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohexane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described by Paulus (Behring Ins. Mitt. (1985) No. 78, 118-132); Brennan et al. (Science (1985) 229:81-83), and Glennie et al. (J. Immunol. (1987) 139: 2367-2375). Preferred conjugating agents are SATA and sulfo-SMCC, both available from Pierce Chemical Co. (Rockford, Ill.).

When the binding specificities are antibodies (e.g., two humanized antibodies), they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific and multispecific molecule is a MAb×MAb, MAb×Fab, Fab×F(ab′)₂ or ligand×Fab fusion protein. A bispecific and multispecific molecule of the invention, e.g., a bispecific molecule can be a single chain molecule, such as a single, chain bispecific antibody, a single chain bispecific molecule comprising one single chain antibody and a binding determinant, or a single chain bispecific molecule comprising two binding determinants. Bispecific and multispecific molecules can also be single chain molecules or may comprise at least two single chain molecules. Methods for preparing bi- and multispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific and multispecific molecules to their specific targets can be confirmed by enzyme-linked immunosorbent assay (ELISA), a radioimmunoassay (RIA), FACS analysis, a bioassay (e.g., growth inhibition), or a Western Blot Assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

IV. Antibody Conjugates/Immunotoxins

In another aspect, the present invention features a human anti-heparanase monoclonal antibody, or a fragment thereof, conjugated to a therapeutic moiety, such as a cytotoxin, a drug or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other examples of therapeutic cytotoxins that can be conjugated to an antibody of the invention include calicheamicins and duocarmycins. An antibody of the present invention can be conjugated to a radioisotope, e.g., radioactive iodine, to generate cytotoxic radiopharmaceuticals for treating a heparanase-related disorder, such as a cancer.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., “The Preparation And Cytotoxic Properties Of Antibody-Toxin Conjugates”, Immunol. Rev., 62:119-58 (1982), European Patent Number EP 0 624 377 and U.S. Pat. No. 6,214,345.

V. Pharmaceutical Compositions

In another aspect, the present invention provides a composition, e.g., a pharmaceutical composition, containing one or a combination of human monoclonal antibodies, or antigen-binding portion(s) thereof, of the present invention, formulated together with a pharmaceutically acceptable carrier. In a specific embodiment, the compositions include a combination of multiple (e.g., two or more) isolated human antibodies or antigen-binding-portions thereof of the invention. Preferably, each of the antibodies or antigen-binding portions thereof of the composition binds to a distinct, pre-selected epitope of heparanase.

In one embodiment, human anti-heparanase monoclonal antibodies having complementary activities are used in combination, e.g., as a pharmaceutical composition, comprising two or more human anti-heparanase monoclonal antibodies. For example, a human monoclonal antibody that mediates tumor cell migration can be combined with another human monoclonal antibody that inhibits the growth of tumor cells.

In another embodiment, the composition comprises one or a combination of bispecific or multispecific molecules of the invention (e.g., which contains at least one binding specificity for an Fc receptor and at least one binding specificity for heparanase).

Pharmaceutical compositions of the invention also can be administered in combination therapy, i.e., combined with other agents. For example, the combination therapy can include a composition of the present invention with at least one anti-tumor agent or other conventional therapy.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the antibodies of the present invention are administered as pharmaceuticals, to humans and animals, they can be given alone or as a pharmaceutical composition containing, for example, 0.01 to 99.5% (more preferably, 0.1 to 90%, 0.1 to 70%, most preferably from 1 to 30%) of active ingredient in combination with a pharmaceutically acceptable carrier.

Regardless of the route of administration selected, the antibodies of the present invention, and/or the pharmaceutical compositions of the present invention, are formulated into pharmaceutically acceptable dosage forms by conventional methods known to those of skill in the art.

The formulations may conveniently be presented in unit dosage form and may be prepared by any methods known in the art of pharmacy. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhancing targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the target e.g., a tumor. The composition must be fluid to the extent that easy syringability exists. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. It must be stable under the conditions of manufacture and storage and must be preserved against the contaminating action of microorganisms such as bacteria and fungi. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

A “therapeutically effective dosage” preferably inhibits tumor growth by at least about 20%, more preferably by at least about 40%, even more preferably by at least about 60%, and still more preferably by at least about 80% relative to untreated subjects. The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

When the active compound is suitably protected, as described above, the compound may be orally administered, for example, with an inert diluent or an assimilable edible carrier.

VI. Uses and Methods of the Invention

Human monoclonal antibodies of the present invention (and derivatives/conjugates thereof) have several in vitro and in vivo diagnostic and therapeutic utilities. For example, the antibodies contacted with cells in culture, e.g. in vitro or ex vivo, or administered to a subject, e.g., in vivo, to treat, prevent or diagnose a variety of disorders. As used herein, the term “subject” is intended to include human and nonhuman animals. The term “nonhuman animals” of the invention includes all vertebrates, e.g., mammals and non-mammals, such as nonhuman primates, sheep, dog, cow, chickens, amphibians, reptiles, etc. Preferred human animals include a human patient having disorder characterized by expression, typically aberrant expression (e.g., over expression) of heparanase. In one embodiment, the antibodies of the present invention can be used to treat a subject with a tumorigenic disorder, e.g., a disorder characterized by the presence of tumor cells, for example, melanoma cells, lymphoma cells, prostate carcinoma cells, pancreatic carcinoma cells, bladder carcinoma cells, fibrosarcoma cells, rhabdomyosarcoma cells, mastocytoma cells, mammary adenocarcinoma cells, leukemia cells, and rheumatoid fibroblasts.

Human antibodies of the invention have additional utility in therapy and diagnosis of heparanase-related diseases. For example, the human antibodies can be used to elicit in vivo or in vitro one or more of the following biological activities: to inhibit activity of human heparanase; to inhibit tumor cell growth and/or motility induced by human heparanase; to opsonize a cell expressing heparanase; or to mediate phagocytosis or cytolysis of a cell expressing heparanase in the presence of human effector cells.

Human antibodies of the present invention can also be used to ameliorate wound healing and to treat inflammatory and autoimmune disorders, such as arthritis (e.g., rheumatoid arthritis or osteoarthritis), asthma, lupus erythematosus, or allograft rejection, as well as disorders such as vascular restenosis, atherosclerosis, fibro-proliferative disorders, or Alzheimer's Disease.

The present invention therefore provides methods of treatment of heparanase-related diseases, such as those recited above, comprising administering a human antibody of the invention; as well as the use of the human antibodies of the invention in the manufacture of a medicament for the treatment of such diseases. For example, the antibodies of the invention can be used in the treatment of melanoma, lymphoma, prostate cancer, pancreatic cancer, bladder cancer, fibrosarcoma, rhabdomyosarcoma, mastocytoma, breast cancer, adenocarcinoma, or leukemia.

Human antibodies of the invention can be initially tested for binding activity associated with therapeutic or diagnostic use in vitro. For example, compositions of the invention can be tested using the ELISA and flow cytometric assays described in the Examples below. Moreover, the activity of these molecules in triggering at least one effector-mediated effector cell activity, including cytolysis of cells expressing heparanase can be assayed. Protocols for assaying for effector cell-mediated phagocytosis are described in the Examples below.

In a particular embodiment, the human anti-heparanase antibodies are co-administered with another therapeutic agent, e.g., a chemotherapeutic agent or an immunosuppressive agent, or co-administered with other known therapies, e.g., an anti-cancer therapy, e.g., radiation. Such therapeutic agents include, among others, anti-nepotistic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Immunosuppressive agents include cyclosporine and anti-inflammatory agents. The chemotherapeutic agent cisplatin is intravenously administered as a 100 mg/m² dose once every four weeks and adriamycin is intravenously administered as a 60-75 mg/m² dose once every 21 days. Co-administration of the human anti-heparanase antibodies of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

The compositions (e.g., human antibodies, multispecific and bispecific molecules) of the invention which have complement binding sites, such as portions from IgG1, -2, or -3 or IgM which bind complement, can also be used in the presence of complement. In one embodiment, ex vivo treatment of a population of cells comprising target cells with an antibody of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

Human antibodies of the invention can also be administered to a subject together with complement. Accordingly, within the scope of the invention are compositions comprising human antibodies, multispecific or bispecific molecules and serum or complement. These compositions are advantageous in that the complement is located in close proximity to the human antibodies. Alternatively, the human antibodies and the complement or serum can be administered separately.

Also within the scope of the invention are kits comprising human antibodies of the invention and, optionally, instructions for use. The kit can further contain a least one additional reagent, such as complement, or one or more additional human antibodies of the invention (e.g., a human antibody having a complementary activity and/or which binds to an epitope in heparanase antigen distinct from the first human antibody).

In other embodiments, the subject can be additionally treated with an agent that modulates, e.g., enhances or inhibits, the expression or activity of Fcγ or Fcα receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

In another embodiment, human antibodies of the invention can be used to detect the presence of heparanase antigen in a sample, or measure the amount of heparanase antigen, by contacting the sample, and a control sample, with the antibody under conditions that allow for formation of a complex between the antibody or portion thereof and heparanase. The formation of a complex is then detected, wherein a difference complex formation between the sample compared to the control sample is indicative the presence of heparanase antigen in the sample.

The present invention is further illustrated by the following examples which should not be construed as further limiting. The contents of all figures and all references, patents and published patent applications cited throughout this application are expressly incorporated herein by reference.

### EXAMPLES

Materials and Methods

### Cloning of Heparanase Subunits was Performed as Described in Example 1

Insect cell expression: Baculovirus transfer vectors containing heparanase subunits were co-transfected, along with baculogold DNA, into sf9 insect cells grown in SF900II protein free media (Gibco BRL) using standard calcium phosphate conditions (Pharmingen). Recombinant viruses were plaque purified and Hi titre (10⁸ pfu/ml) virus transfected into Tni cells at an MOI of 2 and harvested at 48 hrs post infection. Cells were pelleted at 1000 g for 10 minutes at 4° C. and crude supernatants containing heparanase kept at 4° C. until purification.

Purification and detection of recombinant heparanase: Routinely 1.5 litre batches of secreted recombinant heparanase enzyme in SF900II media was passed over a 5 ml heparin sepharose Hi-Trap column (Pharmacia) using a FPLC system. Enzyme was loaded at 1 ml/min, column washed in 10 column volumes of PBS, and then eluted with a gradient of 1M NaCl in 25 mM Tris pH 7.5, collecting 5 ml fractions. Typically heparanase elutes at 0.67M NaCl in this system and produces yields of around 1 mg/litre. Heparanase was concentrated and buffer exchanged by centrifuging through a 30 kDa cut-of f filter and was routinely >90% pure as assessed by SDS PAGE gels.

Heparanase enzyme activity (³⁵S ECM method): Measurements of heparanase enzyme activities were carried out as described previously (Vlodavsky, I. et al. (1999) Mammalian heparanase: gene cloning, expression and function in tumor progression and metastasis. Nature Med. 5: 793-802). Heparanase was incubated for 18 h at 37° C., pH 6.2, with ³⁵S labeled heparin sulphate and the supernatant was analyzed by gel filtration on a sepharose CL-6B column (0.9×30 cm). Fractions were eluted with PBS and their radioactivity measured.

Heparanase enzyme inhibition ELISA: A 96-well plate was coated overnight at room temperature with 100 μl/well of heparan sulphate in PBS. The wells were aspirated, blocked for 1 hour with 200 μl/well 1% BSA/PBS, and then washed five times with 200 μl/well 0.05% Tween 20/PBS. 100 μl/well of recombinant human basic FGF (90 ng/ml in 0.1% BSA/PBS) was then added and the plate incubated at 37° C. for 1 hour. The plate was then washed, 100 μl buffer (1% BSA/PBS) or heparanase was added with or without monoclonal antibodies, and the plate incubated for 2 hours at 37° C. shaking. After washing, 100 μl/well of anti-basic FGF HRP conjugate was added and incubated at 37° C for 1 hour. The plate was again washed, 100 μl of TMB added for 10 minutes and the reaction stopped with 50 μl 1M H₂SO₄ and the plate read at OD450.

Cell motility/invasion assay: 0.75 ml of media (RPMI 1640 plus 2 mM glutamine) with 0.1% UltraserG (Gibco) and 25 μg/ml Fibronectin was added into each well to be used of a 24 well plate. A chamber insert (with 8 μM pore size) was then placed into each well. PC3 cells in logarithmic growth phase were collected, washed with PBS, the supernatant aspirated, and resuspended in media containing 0.1% UltraserG. The cells were then plated in the upper chamber at a density of 1×10⁵ cells per well in a volume of 180 μl with or without monoclonal antibodies and controls (20 μl). Cells that migrated through to the bottom chamber were counted (3 random fields per well) after a 24 hour incubation in a 37° C. incubator.

BIAcore assay: Surface plasmon resonance (BIAcore 3000) measurement was used to determine the affinity constant of human monoclonal antibody 22D9. The run buffer included 10 mM HEPES, pH 7.4 containing 150 mM NaCl, 3.4 mM EDTA, and 0.01% surfactant P20. Protein A (approximately 1000 RU) was immobilized to the sensor chip surface using the amine coupling kit (BIAcore). Using a flow rate of 30 ul/min, human monoclonal antibody 22D9 was first bound to the Protein A (approximately 200RU) followed by injections of heparanase. Buffer flow and regeneration of the sensor chip surface with 100 mM HCl followed. BIAcore software was utilized for association and dissociation phase analysis.

In vivo study: A co-injection model using heparanase transfected MDA-MB 435 cells (a human breast cancer line derived from a ductal carcinoma) was used to test the efficacy of human monoclonal antibody 22D9. Cells were implanted in both flanks (2×10⁶/flank) of a nu/nu mouse and antibody treatment started the same day at a dose of 0.2 mg/mouse by the intravenous route (tail vein). Treatment continued every 4 days at the same dose for a total of 10 doses. The animals were sacrificed when tumor size or other clinical signs necessitated removal of the mouse from the study.

### Example 1

### Cloning and Expression of Active Heparanase Heterodimer Enzyme

Heparanase exists as a 65 kDa precursor which is subsequently cleaved by protease action to liberate a heterodimeric protein consisting of non-covalently associated 8 kDa and 50 kDa subunits (Fairbanks, M. B. et al. (1999) Processing of the human heparanase precursor and evidence that the active enzyme is a heterodimer. J. Biol. Chem. 274, 29587-29590). The enzyme can be naturally secreted and, thus, the 8 kDa and 50 kDa subunits were co-expressed in baculovirus secretory vectors. Both individual subunits (8 and 50 kDa subunits) were initially amplified by PCR from a mammary gland cDNA library using the primers and conditions described below and cloned into pZero Blunt before excision and cloning into pAcGP67 vectors. Cloning into pAcGP67 generated an additional four amino acids at the N-termini of both subunits which had no detrimental effect on the enzymatic activity. 8 kDa Forward: 5′ CCC GGG CAG GAC GTC GTG GAC CTG GAC TTC TTC ACC 3′ (SEQ ID NO:55); 8 kDa Reverse: 5′ GAA TTC TCA TTC CTT CTT GGG ATC GAA AAT TAG GAA 3′ (SEQ ID NO:56). 50 kDa Forward: 5′ CCC GGG AAA AAG TTC AAG (SEQ ID NO:57); 50 kDa Reverse: 5′ GAA TTC TCA GAT GCA AGC AGC AAC TTT GGC ATT TCT 3′ (SEQ ID NO:58). Cycling conditions were 94° C. for 1 minute, (94° C. for 30 seconds, 60° C. for 30 seconds, 72° C. for 2 minutes) 30 cycles, 4° C.

For construction of the dual expression construct, the cDNAs for the two subunits, along with flanking GP67 secretory sequence (sequence ADPG encoded by 5′ GCG GAT CCC GGG 3′(SEQ ID NO:59) AAC AGC ACC TAC TCA AGA 3′ (SEQ ID NO:60)), were PCR amplified from pAcGP67A: heparanase templates using the primers: Dual 8 kDa Forward; 5′ AGA TCT ATG CTA CTA GTA AAT CAG TCA CAC CAA GGC 3′ (SEQ ID NO:61); Dual 8 kDa Reverse: 5′ AGA TCT TCA TTC CTT CTT GGG ATC GAA AAT TAG GAA 3′ (SEQ ID NO:62). Dual 50 kDa Forward: 5′ GAA TTC ATG CTA CTA GTA AAT CAG TCA CAC CAA GGC 3′ (SEQ ID NO:63); Dual 50 kDa Reverse; 5′ GAA TTC TCA GAT GCA AGC AGC AAC TTT GGC ATT TCT 3′ (SEQ ID NO:64). Both PCR products were cloned into pZeroBlunt and excised with appropriate enzymes; EcoR1 (50 kDa) and Bgl11 (8 kDa). Restriction products were then cloned individually into appropriately digested pAcUW51 (Pharmingen). The ability of the individual subunits of heparanase and the heterodimer generated to digest heparan sulfate (HS) was determined by a ³⁵S-labelled HS assay. Neither the 8 kDa nor the 50 kDa subunits alone were able to digest the HS substrate and eluted labeled fragments were of a similar size to those of free undigested substrate. In contrast, the addition of the 8/50 heterodimer protein resulted in a size shift towards small molecular weight eluted fragments indicating a high level of enzymatic activity. Whole cell extract material was also prepared from PC3M cell lines and incubated, as described above, with the radiolabeled HS to compare the digestion profile obtained from mammalian expressed enzyme. The profiles were completely super-imposable on each other indicating that recombinantly produced enzyme cleaves high molecular weight HS into fragments indistinguishable from that produced by the native enzyme.

### Example 2

### Production of Human Antibodies Against Heparanase

KM Mice

Fully human monoclonal antibodies to heparanase were prepared using KM mice which express human antibody genes. In this mouse strain, the endogenous mouse kappa light chain gene has been homozygously disrupted as described in Chen et al. (1993) EMBO J. 12:811-820 and the endogenous mouse heavy chain gene has been homozygously disrupted as described in Example 1 of International Patent Application WO 01/09187. This mouse strain carries a human kappa light chain transgene, KCo5, as described in Fishwild et al. (1996) Nature Biotechnology 14:845-851. This mouse strain also carries a human heavy chain transchromosome composed of chromosome 14 fragment hCF (SC20) as described in International Patent Application WO 02/43478.

KM Mice Immunizations

To generate fully human monoclonal antibodies to heparanase, KM mice were immunized with purified recombinant heparanase antigen. General immunization schemes for mice carrying human antibody genes are described in Lonberg, N. et al. (1994) Nature 368(6474): 856-859; Fishwild, D. et al. (1996) Nature Biotechnology 14: 845-851 and International Patent Application WO 98/24884. The mice were 6-16 weeks of age upon the first infusion of antigen. A purified recombinant preparation (25-50 μg) of heparanase antigen (e.g., purified from transinfected baculovirus cells expressing heparanase enzyme) was used to immunize the KM mice intraperitonealy (IP) or subcutaneously (Sc).

Mice were immunized intraperitonealy (IP) or subcutaneously (Sc) with antigen in complete Freund's adjuvant twice, followed by 2-4 weeks IP immunization (up to a total of 7 immunizations) with the antigen in incomplete Freund's adjuvant. The immune response was monitored by retroorbital bleeds. The plasma was screened by ELISA (as described below), and mice with sufficient titers of anti-heparanase human immunoglobulin were used for fusions. Mice were boosted intravenously with antigen 3 and 2 days before sacrifice and removal of the spleen. Typically, 10-15 fusions for were performed and several dozen mice were immunized.

Generation of Hybridomas Producing Human Monoclonal Antibodies to Heparanase:

The mouse splenocytes were isolated from the KM mice fused with PEG to a mouse myeloma cell line based upon standard protocols. The resulting hybridomas were then screened for the production of heparanase-specific antibodies. Single cell suspensions of splenic lymphocytes from immunized mice were fused to one-fourth the number of P3×63-Ag8.653 nonsecreting mouse myeloma cells (ATCC, CRL 1580) or SP2/0 nonsecreting mouse myeloma cells (ATCC, CRL 1581) with 50% PEG (Sigma). Cells were plated at approximately 1×10⁵/well in flat bottom microtiter plate, followed by about two week incubation in selective medium containing 10% fetal bovine serum, 10% P388D1 (ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1× HAT (Sigma, CRL P-7185). After 1-2 weeks, cells were cultured in medium in which the HAT was replaced with HT. Individual wells were then screened by ELISA (described below) for human anti-heparanase monoclonal IgG antibodies. Once extensive hybridoma growth occurred, medium was monitored usually after 10-14 days. The antibody secreting hybridomas were replated, screened again and, if still positive for human IgG, anti-heparanase monoclonal antibodies were subcloned at least twice by limiting dilution. The stable subclones were then cultured in vitro to generate small amounts of antibody in tissue culture medium for characterization.

Selection of Human Monoclonal Antibodies Binding to Heparanase/Primary Screens

To select KM mice producing antibodies that bound heparanase, sera from immunized mice was tested by ELISA as described by Fishwild, D. et al. (1996). Briefly, microtiter plates were coated with purified recombinant heparanase from baculovirus at 1-2 μg /ml in PBS, 50 μl/wells incubated 4° C. overnight then blocked with 200 μl/well of 5% chicken serum in PBS/Tween (0.05%). Dilutions of plasma from heparanase-immunized mice were added to each well and incubated for 1-2 hours at ambient temperature. The plates were washed with PBS/Tween and then incubated with a goat-anti-human IgG Fc polyclonal antibody conjugated with horseradish peroxidase (HRP) for 1 hour at room temperature. After washing, the plates were developed with ABTS substrate (Sigma, A-1 888, 0.22 mg/ml) and analyzed by spectrophotometer at OD 415-495. Mice that developed the highest titers of anti-heparanase antibodies were used for fusions. Fusions were performed as described above and hybridoma supernatants were tested for anti-heparanase activity by ELISA. In order to select antibodies that bind to heparanase enzyme, an ELISA assay as described above and a biotinylated ELISA as described above with following modifications were applied. Briefly, ELISA plates were coated with streptavidin (Sigma catalog number: S-4762) at 1 μg/ml in PBS, 50 ul/well. After overnight incubation at 4° C., the plates were blocked with 200 μl/well of 5% chicken serum in PBS/Tween (0.05%). The plates were then washed with PBS/Tween three times. Biotin-heparanase was added to the plates at 1 μg/ml in PBS, 50 ul/well and then incubated for 1 hour at room temperature. 50 ul/well of the supernatants were then added to the plates and incubated for 1-2 hours at room temperature. The rest of the procedures were exactly the same as described above. Hybridomas secreting a monoclonal antibody that bound with high avidity to heparanase were selected for further characterization for enzymatic, motility and angiogenesis activities.

Clones which bound to heparanase and were positive in enzymatic, motility, and angiogenesis assays were subcloned. One clone from each hybridoma, which retained the reactivity of parent cells (by ELISA), was chosen. 5-10 vial cell banks were generated for each clone and stored in liquid nitrogen.

To determine the isotype of antibodies, an isotype ELISA was performed. Wells of microtiter plates were coated with 1 μg/ml of mouse anti human kappa light chain, 50 μl/well in PBS incubated 4° C. overnight. After blocking with 5% chicken serum, the plates were reacted with supernatant and purified isotype control. Plates were then incubated at ambient temperature for 1-2 hours. The wells were then reacted with either human IgG1, IgG2, IgG3 or IgG4-specific Horseradish peroxidase—conjugated probes. Plates were developed and analyzed as described above.

### Example 3

### MAb Screening Assays

Human monoclonal antibodies that were positive in the primary screening were further selected by secondary screening assays of: inhibition of heparanase activity; inhibition of heparanase driven cell motility; and affinity analysis (BIAcore measurement).

The secondary screening assay measured the inhibition of heparanase activity in the enzyme ELISA assay. Human anti-heparanase IgG from hybridoma samples was purified by Protein A chromatography and buffer exchanged into PBS (phosphate buffered saline). The concentration of heparanase used to screen the hybridoma IgG was 50 ng/ml. Positives were classified as inhibiting heparanase activity by 75% and were further tested in the cell motility assay and by BIAcore for binding to heparanase. IgG prepared from all clones of human monoclonal antibody 22D9 inhibited heparanase in the enzyme ELISA and cell motility assay.

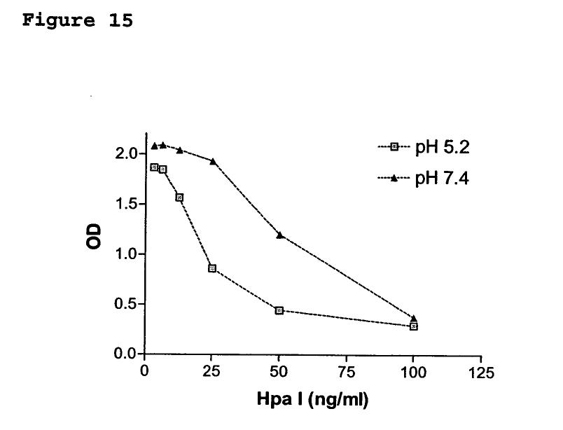

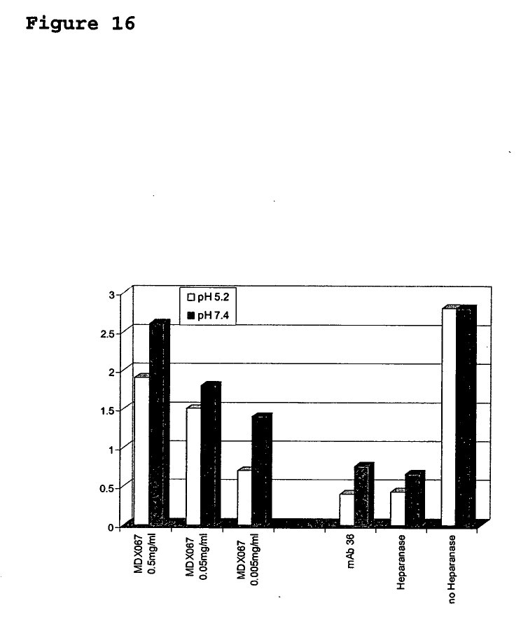

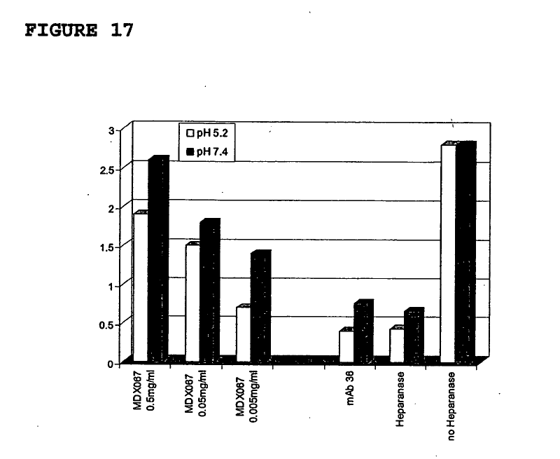

The pH activity profile of heparanase is shown in FIG. 15 which indicates that the maximal activity of the enzyme was observed at pH 5.2. The activity of heparanase at pH5.2 is approximately 3 times greater than heparanase activity at pH 7.4 in this assay format. Human monoclonal antibody 22D9 inhibited heparanase activity in a dose dependent manner at the enzyme's optimal pH 5.2 (FIG. 16). Comparable inhibition of heparanase activity with human monoclonal antibody 22D9 was observed at pH 5.2 and 7.4 (FIG. 17). Heparanase was used at a concentration of 100 ng/ml which dose gives a comparable activity in the enzyme ELISA at pH 5.2 or 7.4. The addition of normal human IgG or a non-neutralizing MAb (MAb 36) at a concentration of 0.15 mg/ml had no effect in this assay (equivalent to the buffer control).

The affinity of 22D9 was determined by BIAcore analysis as follows:

K_{assoc}(M⁻¹s⁻¹)1.8×10⁻⁵

K_{diss}(s)4.6×10⁻⁴

K_{D}(M⁻¹)2.5×10⁹

Studies using antibodies in a competition binding format have demonstrated that MAb 14 (non-neutralizing anti-heparanase antibody) and human monoclonal antibody 22D9 bind to different epitopes. In addition, polyclonal antisera raised against peptides from the 8 kDa subunit of heparanase prevented binding of human monoclonal antibody 22D9 to heparanase (and vice versa). Therefore, human monoclonal antibody 22D9 likely binds to the small subunit or a site on the large subunit that is in close proximity to the small subunit.

### Example 4

### Inhibition of Cell Motility by Human Monoclonal Antibody 22D9

A role for heparanase in cell migration and the metastatic process has been proposed (Vlodavsky, I and Friedman, Y. (2001) Molecular properties and involvement of heparanase in cancer metastasis and angiogenesis. J Clin Invest. 108: 341-7). Therefore, the effect of human monoclonal antibodies which bind to and inhibit heparanase activity in an in vitro motility/migration assay utilizing PC3 cells (a prostate derived line) was investigated.

Briefly, chamber inserts (with 8 μM pore size) were placed into wells containing the appropriate media. PC3 cells in logarithmic growth phase were plated in the upper chamber at a density of about 1×10⁵ cells per well in a volume of about 180 μl with or without monoclonal antibodies and controls (20 μl). Cells that migrate through to the bottom chamber were counted and cell motility determined.

Human monoclonal antibody 22D9 significantly inhibited PC3 cell motility (Table 1) compared to media alone or a non-neutralizing anti-heparanase antibody (MAb 14). The number of PC3 cells that migrated through to the bottom chamber was averaged from 3 views per well (SD<10%). The control consisted of media only.

TABLE 1

| | Number of Cells | Number of Cells |
|---|---|---|
| MAb | Assay 1 | Assay 2 |
| 22D9 | 59 | 10 |
| MAb 14 | 114 | — |
| Control | 131 | 147 |

### Example 5

### Inhibition of Cell Growth by Human Monoclonal Antibody 22D9

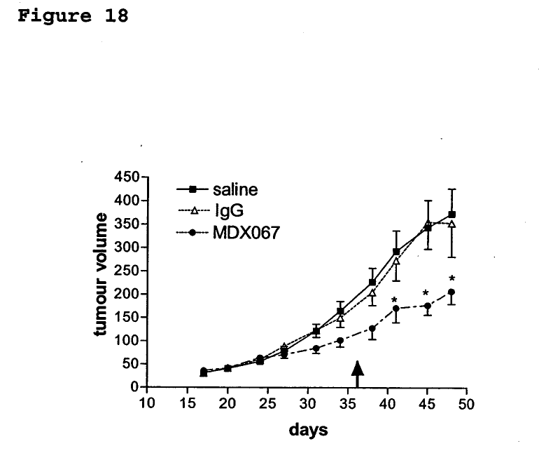

The effect of human monoclonal antibody 22D9 on the growth of MDA-MB 435 cells (breast tumor cell line) in nu/nu mice was investigated (FIG. 18). Cells were injected on both flanks of the mice. Saline, IgG, or human monoclonal antibody 22D9 was administered intravenously at day 0 and at 4 day intervals until day 36 (arrow). Tumor volume was calculated by the following formula: (W²×L)/2 where W is the tumor measurement at the widest point and L is the tumor dimension at the longest point. Data are the mean±SEM, n=10, p<0.05. Tumor volume was significantly inhibited by human monoclonal antibody 22D9 at day 42 until the end of the study (FIG. 18). In total, the tumor volume was reduced by approximately 50%.

### Example 6

### V_{H} and V_{L}—Region Sequencing of Human Monoclonal Antibodies

Human monoclonal antibodies from hybridomas were purified by protein A column chromatography which led to the isolation of five antibodies of interest: 2H8, 22D9, 13B2, 5G10, and 1E7. The nucleotide and deduced amino acid sequence of rearranged V_{H} and V_{L}-domains of human monoclonal antibodies 2H8, 22D9, 13B2, 5G10, and 1E7 were determined using standard procedures. Accordingly, the V_{H} and V_{L} regions of antibodies 2H8, 22D9, 13B2, 5G10, and 1E7 were subsequently isolated from hybridoma RNA and reverse transcribed to cDNA. The V regions were amplified by the polymerase chain reaction (PCR®) and the PCR product was sequenced. The following includes the nucleotide (NA) and amino acid (AA) sequences of the V_{H} and V_{L} regions of each antibody:

| Antibody | V_{H} Region NA | V_{H} Region AA | V_{L} Region NA | V_{L} Region AA |
|---|---|---|---|---|
| 2H8 | SEQ ID NO: 1 | SEQ ID NO: 2 | SEQ ID NO: 3 | SEQ ID NO: 4 |
| 22D9 | SEQ ID NO: 5 | SEQ ID NO: 6 | SEQ ID NO: 7 | SEQ ID NO: 8 |
| 13B2 | SEQ ID NO: 9 | SEQ ID NO: 10 | SEQ ID NO: 11 | SEQ ID NO: 12 |
| 5G10 | SEQ ID NO: 13 | SEQ ID NO: 14 | SEQ ID NO: 15 | SEQ ID NO: 16 |
| 1E7 | SEQ ID NO: 17 | SEQ ID NO: 18 | SEQ ID NO: 19 | SEQ ID NO: 20 |

Equivalents

Those skilled in the art will recognize or be able to ascertain, using no more than routine experimentation, many equivalents of the specific embodiments of the invention described herein. Such equivalents are intended to be encompassed by the following claims.

Incorporation by Reference

All patents, pending patent applications and other publications cited herein are hereby incorporated by reference in their entirety.

We claim:

1. An isolated human monoclonal antibody, or an antigen-binding portion thereof, that has the following properties:
(a) the antibody binds to human heparanase;
(b) the antibody inhibits enzymatic activity of human heparanase;
(c) the antibody inhibits cell motility; and
(d) the antibody inhibits tumor cell growth.

2. The antibody of claim 1, wherein the antibody is selected from the group consisting of an IgG1, an IgG2, an IgG3, and an IgG4 antibody.

3. The antibody of claim 2, wherein the antibody is an IgG1 antibody.

4. The antibody of claim 3, comprising a variable region from an IgG1 heavy chain and a variable region from a kappa light chain.

5. The antibody of claim 1, wherein the antibody binds to human heparanase with an association constant (K_{assoc}) of at least about 10⁻⁴ M⁻¹S⁻¹.

6. The antibody of claim 1, wherein the antibody dissociates from human heparanase with at a rate constant (K_{diss}) of about 10⁻³ S⁻¹ or less.

7. The antibody of claim 1, wherein the antibody binds to human heparanase with an affinity constant (K_{A}) of at least about 10⁷ M⁻¹.

8. An isolated human monoclonal antibody, or antigen binding portion thereof, comprising a human heavy chain variable region comprising CDR1, CDR2, and CDR3 sequences and a human light chain variable region comprising CDR1, CDR2, and CDR3 sequences, wherein:
(a) the human heavy chain variable region CDR3 sequence comprises SEQ ID NO:33, or conservative sequence modifications thereof ;
(b) the human light chain variable region CDR3 sequence comprises SEQ ID NO:36, or conservative sequence modifications thereof ;
(c) the antibody binds to human heparanase;
(d) the antibody inhibits enzymatic activity of human heparanase;
(e) the antibody inhibits cell motility; and
(f) the antibody inhibits tumor cell growth.

9. The antibody of claim 8, wherein the human heavy chain variable region CDR2 sequence comprises SEQ ID NO:32, or conservative sequence modifications thereof ; and the human light chain variable region CDR2 sequence comprises SEQ ID NO:35, or conservative sequence modifications thereof.

10. The antibody of claim 8, wherein the human heavy chain variable region CDR1 sequence comprises SEQ ID NO:31, or conservative sequence modifications thereof ; and the human light chain variable region CDR1 sequence comprises SEQ ID NO:34, or conservative sequence modifications thereof.

11. An isolated human monoclonal antibody comprising:
(a) a heavy chain variable region comprising CDR1, CDR2, and CDR3 sequences comprising SEQ ID NOs:31, 32, and 33, respectively; and
(b) a light chain variable region comprising CDR1, CDR2, and CDR3 sequences comprising SEQ ID NOs:34, 35, and 36, respectively,
wherein the antibody binds to human heparanase.

12. An isolated human monoclonal antibody, or antigen binding portion thereof, comprising a human heavy chain variable region comprising CDR1, CDR2, and CDR3 sequences and a human light chain variable region comprising CDR1, CDR2, and CDR3 sequences, wherein:
(a) the human heavy chain variable region CDR3 sequence comprises an amino acid sequence selected from the group consisting of SEQ ID NOs:27, 39, 45, and 51, and conservative sequence modifications thereof ;
(b) the human light chain variable region CDR3 sequence comprises an amino acid sequence selected from the group consisting of SEQ ID NOs:30, 42, 48, and 54, and conservative sequence modifications thereof ;
(c) the antibody binds to human heparanase;
(d) the antibody inhibits enzymatic activity of human heparanase;
(e) the antibody inhibits cell motility; and
(f) the antibody inhibits tumor cell growth.

13. The antibody of claim 12, wherein the human heavy chain variable region CDR2 sequence comprises an amino acid sequence selected from the group consisting of SEQ ID NOs:26, 38, 44, and 50, and conservative sequence modifications thereof ; and the human light chain variable region CDR2 sequence comprises an amino acid sequence selected from the group consisting of SEQ ID NOs:29, 41, 47, and 53, and conservative sequence modifications thereof.

14. The antibody of claim 12, wherein the human heavy chain variable region CDR1 sequence comprises an amino acid sequence selected from the group consisting of SEQ ID NOs:25, 37, 43, and 49, and conservative sequence modifications thereof ; and the human light chain variable region CDR1 sequence comprises an amino acid sequence selected from the group consisting of SEQ ID NOs:28, 40, 46, and 52, and conservative sequence modifications thereof.

15. An isolated human monoclonal antibody selected from the group consisting of :
(a) an antibody comprising (i) a heavy chain variable region comprising CDR1, CDR2, and CDR3 sequences comprising SEQ ID NOs:25, 26, and 27, respectively; and (ii) a light chain variable region comprising CDR1, CDR2, and CDR3 sequences comprising SEQ ID NOs: 28, 29, and 30, respectively, wherein the antibody binds to human heparanase;
(b) an antibody comprising (i) a heavy chain variable region comprising CDR1, CDR2, and CDR3 sequences comprising SEQ ID NOs:37, 38, and 39, respectively; and (ii) a light chain variable region comprising CDR1, CDR2, and CDR3 sequences comprising SEQ ID NOs: 40, 41, and 42, respectively, wherein the antibody binds to human heparanase;
(c) an antibody comprising (i) a heavy chain variable region comprising CDR1, CDR2, and CDR3 sequences comprising SEQ ID NOs:43, 44, and 45, respectively; and (ii) a light chain variable region comprising CDR1, CDR2, and CDR3 sequences comprising SEQ ID NOs: 46, 47, and 48, respectively, wherein the antibody binds to human heparanase; and
(d) an antibody comprising (i) a heavy chain variable region comprising CDR1, CDR2, and CDR3 sequences comprising SEQ ID NOs:49, 50, and 51, respectively; and (ii) a light chain variable region~comprising CDR1, CDR2, and CDR3 sequences comprising SEQ ID NOs: 52, 53 and 54, respectively, wherein the antibody binds to human heparanase;

16. An isolated human monoclonal antibody, or antigen binding portion thereof, comprising a human heavy chain variable region and a human light chain variable region, wherein:
(a) the human heavy chain variable region comprises an amino acid sequence that is at least 90% homologous to the amino acid sequence of SEQ ID NO:6;
(b) the human light chain variable region comprises an amino acid sequence that is at least 90% homologous to the amino acid sequence of SEQ ID NO:8;
(c) the antibody binds to human heparanase;
(d) the antibody inhibits enzymatic activity of human heparanase;
(e) the antibody inhibits cell motility; and
(f) the antibody inhibits tumor cell growth.

17. An isolated human monoclonal antibody, or antigen binding portion thereof, comprising human heavy chain and human light chain variable regions comprising the amino acid sequences shown in SEQ ID NO:6 and SEQ ID NO:8, respectively.

18. An isolated human monoclonal antibody, or antigen binding portion thereof, comprising a human heavy chain variable region and a human light chain variable region, wherein:
(a) the human heavy chain variable region comprises an amino acid sequence that is at least 90% homologous to an amino acid sequence selected from the group consisting of SEQ ID NOs: 2, 10, 14, and 18;
(b) the human light chain variable region comprises an amino acid sequence that is at least 90% homologous to an amino acid sequence selected from the group consisting of SEQ ID NOs: 4, 12, 16, and 20;
(c) the antibody binds to human heparanase;
(d) the antibody inhibits enzymatic activity of human heparanase;
(e) the antibody inhibits cell motility; and
(f) the antibody inhibits tumor cell growth.

19. An isolated human monoclonal antibody, or antigen binding portion thereof, comprising a human heavy chain variable region comprising an amino acid sequence selected from the group consisting of SEQ ID NOs: 2, 10, 14, and 18 and a human light chain variable region comprising an amino acid sequence selected from the group consisting of SEQ ID NOs: 4, 12, 16, and 20.

20. An isolated human monoclonal antibody comprising:
(a) a heavy chain variable region derived from a human V_{H}1-24 germline sequence (SEQ ID NO:21); and
(b) a light chain variable region derived from a human VκL15 (SEQ ID NO:23) germline sequence;
wherein the human antibody binds human heparanase.

21. An isolated human monoclonal antibody comprising:
(a) a heavy chain variable region derived from a human V_{H}3-07 germline sequence (SEQ ID NO:22); and
(b) a light chain variable region derived from a human VκL18 germline sequence (SEQ ID NO:24);
wherein the human antibody binds to human heparanase.

22. The antibody of claim 1 which is an antibody fragment or a single chain antibody.

23. The antibody of claim 1, wherein the cell is a tumor cell selected from the group consisting of a melanoma cell, a lymphoma cell, a prostate carcinoma cell, a pancreatic carcinoma cell, a bladder carcinoma cell, a mast cell, a fibrosarcoma cell, a rhabdomyosarcoma cell, a mastocytoma cell, a mammary adenocarcinoma cell, a leukemia cell, and a rheumatoid fibroblast.

24. A hybridoma comprising a B cell obtained from a transgenic nonhuman animal having a genome comprising a human heavy chain transgene and a light chain transgene fused to an immortalized cell, wherein the hybridoma produces a detectable amount of the antibody of claim 1.

25. A transfectoma comprising nucleic acids encoding a human heavy chain and a human light chain, wherein the transfectoma produces a detectable amount of the antibody of claim 1.

26. A transgenic nonhuman animal which expresses the antibody of claim 1, wherein the transgenic nonhuman animal has a genome comprising a human heavy chain transgene or transchromosome and a human light chain transgene.

27. A method of producing the antibody of claim 1, comprising:
immunizing a transgenic nonhuman animal having a genome comprising a human heavy chain transgene and a human light chain transgene with human heparanase or a cell expressing human heparanase, such that antibodies are produced by B cells of the animal;
isolating B cells of the animal;
fusing the B cells with myeloma cells to form immortal, hybridoma cells that secrete human monoclonal antibodies specific for heparanase; and
isolating the human monoclonal antibodies specific for heparanase from the culture supernatant of the hybridoma.

28. A composition comprising the antibody of claim 1 and a pharmaceutically acceptable carrier.

29. An immunotoxin comprising the antibody of claim 1 linked to a cytotoxic agent.

30. A method of inhibiting motility or growth of a tumor cell, comprising contacting the cell with an effective amount of the antibody of claim 1 such that the growth of the cell is inhibited.

31. A method of treating or preventing a disease mediated by expression of heparanase, comprising administering to a subject the antibody of claim 1 in an amount effective to treat or prevent the disease.

32. A method for detecting the presence of heparanase antigen, or a cell expressing heparanase, in a sample comprising contacting the sample with the antibody of claim 1 under conditions that allow for formation of a complex between the antibody, and heparanase; and detecting the formation of a complex.

33. An expression vector encoding the antibody of claim 1, comprising a nucleotide sequence encoding the variable region of a light chain, heavy chain or both light and heavy chains of a human antibody which binds human heparanase.
